(19) United States
(12) Patent Application Publication
(10) Pub. No.: US 2005/0163844 A1
(43) Pub. Date: Jul. 28, 2005

(54) Controlled and sustained delivery of nucleic acid-based therapeutic agents

(75) Inventor: Paul Ashton, Boston, MA (US)

Correspondence Address: FISH & NEAVE IP GROUP;ROPES & GRAY LLP, ONE INTERNATIONAL PLACE, BOSTON, MA, 02110-2624, US

(73) Assignee: Control Delivery Systems, Inc., Watertown, MA (US)

(21) Appl. No.: 11/043,884

(22) Filed: Jan. 26, 2005

Related U.S. Application Data
(60) Provisional application No. 60/539,293, filed on Jan. 26, 2004.

(51) Int. Cl. A61K 48/00; C12N 15/85; C12N 15/87; A61K 9/22

(52) U.S. Cl. 424/468; 514/44; 435/455

(57) ABSTRACT

The invention provides insertable drug delivery devices for the controlled and sustained release of nucleic acid-based therapeutic agents, including antisense agents, siRNAs, ribozymes, and aptamers.

### RELATED APPLICATION

This application claims the benefit of U.S. Provisional Application No. 60/539,293, filed Jan. 26, 2004, the specification of which is hereby incorporated by reference in its entirety.

### BACKGROUND OF THE INVENTION

There are a number of nucleic acid-based therapeutic agents in various stages of development at this time. Among them are antisense agents, aptamers, ribozymes, and small interfering RNAs (siRNAs). M. Faria, H. Ulrich, Curr. Cancer Drug Targets 2002, 2: 355-368.

Antisense agents are the most advanced class of these agents, with one product (fomivirsen) on the market for the treatment of CMV retinitis, another (alicaforsen) in advanced clinical trials for treatment of Crohn's disease, and Genasense™ (oblimersen sodium), Affinitac™, and Oncomyc-NG™ in clinical trials for treatment of cancer. Antisense agents are typically short, chemically-modified oligonucleotide chains that hybridize to a specific complementary area of a targeted mRNA. The resulting mRNA duplex is recognized and degraded by RNAse H, thereby destroying the mRNA. Because the mRNA instructions fail to reach the ribosome, production of the protein encoded by the targeted mRNA is prevented. By inhibiting the production of proteins involved in disease, antisense drugs can produce a therapeutic benefit.

An aptamer is a DNA or RNA molecule that has been selected from a random or biased pool of oligonucleic acids, based on its ability to bind to a target molecule. Aptamers can be selected which bind nucleic acids, proteins, small organic compounds and specific cell surfaces, and several have been developed which bind to proteins which are associated with disease states. Aptamers are in general more easily manufactured and are more amenable to chemical modification than are antibodies, and they can be “evolved” for tighter binding to the target by an iterative process of random modification and affinity-based selection. The evolved aptamers often have antibody-like specificities, and are therefore expected to have utility in those applications, such as therapeutics and in vitro and in vivo diagnostics, where antibodies have already proved useful. At least one product, Macugen™ (pegaptanib sodium, a PEGylated aptamer with high affinity for VEGF), is in advanced clinical trials for the treatment of age-related macular degeneration.

Ribozymes, or RNA enzymes, are RNA molecules that can catalyze a chemical reaction. All ribozymes found naturally so far catalyze the cleavage of RNA. They range in size from the large “hammerhead” ribozymes to the so-called “minizymes” which are synthetic constructs containing the minimal structures needed for activity. DNA-based enzymes (deoxyribozymes, or DNAzymes) having similar properties have also been prepared. The ability of ribozymes to recognize and cut specific mRNA molecules gives them considerable potential as therapeutic agents. A ribozyme designed to catalyze the cleavage of a specific mRNA would be useful as a therapeutic agent in the same way that a complimentary antisense nucleic acid would be, but with the advantage that a single ribozyme molecule can destroy many copies of the mRNA. A synthetic ribozyme (Angiozyme™) that cleaves the mRNA encoding a VEGF receptor subtype is currently in clinical trials for treatment of cancer.

RNA interference (RNAi) is the phenomenon of gene-specific post-transcriptional silencing by double-stranded RNA oligomers (Elbashir et al. Nature 2001, 411: 494-498; Caplen et al., Proc. Natl. Acad. Sci. U.S.A. 2001, 98: 9742-9747). Small inhibitory RNAs (siRNAs), like antisense oligonucleic acids and ribozymes, have the potential to serve as therapeutic agents by reducing the expression of harmful proteins. The double-stranded siRNA is recognized by a protein complex (the RNA induced silencing complex), which strips away one of the strands, facilitates hybridization of the remaining strand to the target mRNA, and then cleaves the target strand. DNA-based vectors capable of generating siRNA within cells are also of interest for the same reason, as are short hairpin RNAs that are efficiently processed to form siRNAs within cells. siRNAs capable of specifically targeting endogenously and exogenously expressed genes have been described; see for example Paddison et al., Proc. Natl. Acad. Sci. U.S.A., 2002, 99: 1443-1448; Paddison et al., Genes & Dev. 2002, 16: 948-958; Sui et al. Proc. Natl. Acad. Sci. U.S.A. 2002, 8: 5515-5520; and Brummelkamp et al., Science 2002, 296: 550-553.

Methods for the administration of nucleic acid-based therapeutics are largely confined to injection techniques, due to the rapid degradation of nucleic acids upon oral administration, poor absorption due to their large size and substantial ionic charge, and the negligible ability of nucleic acids to penetrate skin or mucosal membranes. It is generally agreed that effective delivery of nucleic acid-based therapeutics is a major obstacle to their successful use in medicine (C. Henry, Chem. Eng. News, December 2003, 32-36). For example, if only 1% of an administered dose of a nucleic acid-based therapeutic is taken up by a patient's cells, one would have to administer 100 times the volume, or administer 100 such injections, in order to achieve the cellular uptake of 100% of that initial dose. Especially where repeated dosing is required, injection methods cause problems with patient compliance, and intravenous injections require the intervention of trained medical personnel and the attendant costs.

There is accordingly a need for a method of administration of nucleic acid-based therapeutic agents that does not rely on repeated injections, yet can provide for consistent and prolonged dosing with such agents over a prolonged period of time.

### SUMMARY OF THE INVENTION

One embodiment of the present invention provides a drug delivery device suitable for the controlled and sustained release of one or more nucleic acid-based therapeutic agents that are effective in obtaining desired local or systemic physiological and/or pharmacological effects.

The device may comprise an inner drug core comprising an amount of a nucleic acid-based therapeutic agent, and a first polymer coating partially covering said core, the polymer coating being impermeable to the therapeutic agent. The device may further comprise a second polymer coating covering at least the portion of the core not covered by the first polymer layer, the second polymer coating being permeable to the therapeutic agent.

The second polymer layer may lie between the core and the first polymer layer, or in alterative embodiments the first polymer layer may lie between the core and the second polymer layer.

Another embodiment provides a method for treating patients, including but not limited to human patients, to obtain a desired local or systemic physiological and/or pharmacological effect. The method comprises positioning a controlled- and sustained-release drug delivery device containing one or more nucleic acid-based therapeutic agents in an area where release of the agent(s) is desired, and allowing the agent(s) to pass from the device to the desired area of treatment.

The drug delivery systems of the present invention may be inserted into any desired area of the body, including but not limited to intradermal, intramuscular, intraperitoneal, intraocular, or subcutaneous sites. Insertion may be achieved by methods including but not limited to injection and surgical implantation.

Nucleic acid-based therapeutic agents suitable for use in the present invention include, but are not limited to, fomivirsen, alicaforsen, oblimersen, pegaptanib, Angiozyme™, Affinitac™, and Oncomyc-NG™.

### BRIEF DESCRIPTION OF THE FIGURES

FIG. 1 is an enlarged cross-sectional illustration of one embodiment of a controlled- and sustained-release drug delivery device in accordance with the present invention.

FIG. 2 is an enlarged cross-sectional illustration of a second embodiment of a controlled- and sustained-release drug delivery device in accordance with the present invention.

FIG. 3 is an enlarged cross-sectional illustration of a third embodiment of a controlled- and sustained-release drug delivery device in accordance with the present invention.

FIG. 4 is a cross-sectional illustration of the embodiment illustrated in FIG. 2, taken at line 4-4.

FIG. 5 schematically illustrates an embodiment of a method in accordance with the present invention of fabricating a drug delivery device.

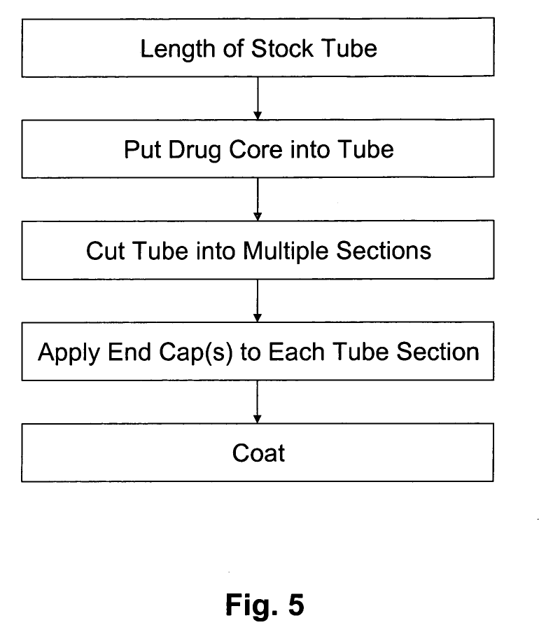

### DETAILED DESCRIPTION OF THE INVENTION

As used herein, the term “nucleic acid” refers to polynucleotides such as deoxyribonucleic acid (DNA), and, where appropriate, ribonucleic acid (RNA). The term should also be understood to include, as appropriate to the context or as applicable to the embodiment being described, both single-stranded polynucleotides (such as antisense) and double-stranded polynucleotides (such as siRNAs).

The term “nucleic acid-based therapeutic agent” as used herein refers to three classes of compounds. The term also includes pharmaceutically acceptable salts, esters, prodrugs, codrugs, and protected forms of the compounds, analogs and derivatives described below. The first class, referred to herein collectively as “antisense nucleic acids,” comprises nucleic acids, preferably oligomers of about 50 monomer units or fewer, which have the ability to hybridize in a sequence-specific manner to a targeted single-stranded RNA or DNA molecule. Members of this class include ordinary DNA and RNA oligomers, DNA and RNA having modified backbones, including but not limited to phosphorothioates, phosphorodithioates, methylphosphonates, and peptide nucleic acids, 2′-deoxy derivatives, and nucleic acid oligomers that feature chemically modified purine and pyrimidine bases, or have been lipophilically modified and/or PEGylated to modify their pharmacodynamics. Oligomers that serve as precursors for such agents, such as hairpin RNAs that are converted to siRNAs within cells, are also considered to be within this class.

The second class of nucleic acid-based therapeutic agents, referred to herein as “aptamers,” comprises nucleic acids, preferably oligomers of about 50 monomer units or fewer, which have the ability to bind with structural specificity to a non-oligonucleotide target molecule, or to an oligonucleotide in a manner other than through sequence-specific hybridization. Members of this class include DNA and RNA aptamers, and modifications thereof including but not limited to mirror-image DNA and RNA (“Spiegelmers”), peptide nucleic acids, and nucleic acid oligomers that have otherwise been chemically modified as described above. Again, any of these species may also feature chemically modified purines and pyrimidines or may be lipophilically modified and/or PEGylated. See M. Rimmele, Chembiochem. 2003, 4: 963-71 and A. Vater and S. Klussmann, Curr. Opin. Drug Discov. Devel. 2003, 6: 253-61 for recent reviews of aptamer technology. It will be appreciated that many members of this second class will, in addition to their structure-specific affinity for the target molecule, have sequence-specific affinity for a putative DNA or RNA sequence.

The third class of nucleic acid-based therapeutic agents, referred to herein as “nucleic acid enzymes,” comprises nucleic acids that are capable of recognizing and catalyzing the cleavage of target RNA molecules, in a sequence-specific manner. The class includes hammerhead ribozymes, minimized hammerheads (“minizymes”), ′10-23′ deoxyribozymes (“DNAzymes”), and the like. As with antisense and aptamer molecules, the class includes catalytic species that have been chemically modified.

The term “pharmaceutically acceptable salts” refers to physiologically and pharmaceutically acceptable salts of the compounds of the invention, e.g., salts that retain the desired biological activity of the parent compound and do not impart undesired toxicological effects thereto.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

By “recombinant virus” is meant a virus that has been genetically altered, e.g., by the addition or insertion of a heterologous nucleic acid construct into the particle.

As used herein, the term “RNAi construct” is a generic term including siRNA, hairpin RNA, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can be converted into siRNAs in vivo.

As used herein, the term “transfection” is art-recognized and means the introduction of a nucleic acid, e.g., an expression vector, into a recipient cell by nucleic acid-mediated gene transfer. “Transformation,” as used herein, refers to a process in which a cell's genotype is changed as a result of the cellular uptake of exogenous DNA or RNA, and, for example, the transformed cell expresses an RNAi construct. A cell has been “stably transfected” with a nucleic acid construct when the nucleic acid construct is capable of being inherited by daughter cells. “Transient transfection” refers to cases where exogenous DNA does not integrate into the genome of a transfected cell, e.g., where episomal DNA is transcribed into mRNA and translated into protein.

As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an episomal vector, e.g., a nucleic acid capable of extra-chromosomal replication. Vectors capable of directing the expression of genes to which they are operatively linked are referred to herein as “expression vectors.” In the present specification, “plasmid” and “vector” are used interchangeably unless otherwise clear from the context. In the expression vectors, regulatory elements controlling transcription can be generally derived from mammalian, microbial, viral or insect genes. The ability to replicate in a host, usually conferred by an origin of replication, and a selection gene to facilitate recognition of transformants may additionally be incorporated. Vectors derived from viruses, such as retroviruses, adenoviruses, and the like, may be employed.

As used herein, even if not particularly called out, the term “insert” means insert, inject, implant, or administer in any other fashion. The term “inserted” means inserted, injected, implanted, or administered in any other fashion. The term “insertion” means insertion, injection, implantation, or administration in any other fashion. Similarly, the term “insertable” means insertable, injectable, implantable, or otherwise administrable.

Permeability is necessarily a relative term. As used herein, the term “impermeable” is intended to mean that the coating, layer, membrane, tube, etc. reduces the release rate of the nucleic acid-based therapeutic agent by at least 70%, preferably by at least 80%, and more preferably by from 90% to about 100%. As used herein, the term “permeable” is intended to mean that the coating, layer, membrane, tube, etc. reduces the release rate of the nucleic acid-based therapeutic agent by no more than 10%. The term “semi-permeable” is intended to mean selectively permeable to at least one substance but not others. It will be appreciated that in certain cases, a membrane may be permeable to a nucleic acid-based therapeutic agent, and also substantially control the rate at which the agent diffuses or otherwise passes through the membrane. Consequently, a permeable membrane may also be a release-rate-limiting or release-rate-controlling membrane, and in certain circumstances, the permeability of such a membrane may be one of the most significant characteristics controlling the release rate for the device. According to an exemplary embodiment of the present invention, a controlled and sustained release drug delivery device comprises an inner reservoir comprising a therapeutically effective amount of a nucleic acid-based therapeutic agent, an inner tube impermeable to the passage of said agent, said inner tube having first and second ends and covering at least a portion of said inner reservoir, said inner tube being dimensionally stable, an impermeable member positioned at said inner tube first end, said impermeable member preventing passage of said agent out of said reservoir through said inner tube first end, and a permeable member positioned at said inner tube second end, said permeable member allowing diffusion of said agent out of said reservoir through said inner tube second end. These and other suitable devices are described in U.S. Pat. No. 6,375,972 and U.S. patent application Ser. No. 10/096,877, the contents of which are incorporated by reference herein in their entirity.

According to another exemplary embodiment, a controlled and sustained release drug delivery device comprises a drug core comprising a therapeutically effective amount of a nucleic acid-based therapeutic agent, a first polymer coating permeable to the passage of said agent, and a second polymer coating impermeable to the passage of said agent, wherein the second polymer coating covers a portion of the surface area of the drug core and/or the first polymer coating. These and other suitable devices are described, for example, in U.S. Pat. No. 5,902,598, the contents of which are incorporated by reference herein in their entirity.

According to another embodiment, a method for providing controlled and sustained administration of a nucleic acid-based therapeutic agent effective in obtaining a desired local or systemic physiological or pharmacological effect comprises inserting a controlled and sustained release drug delivery device of the present invention at a desired location.

According to yet another embodiment, a method of manufacturing a controlled and sustained release drug delivery device comprises manufacturing a drug core containing a nucleic acid-based therapeutic agent, coating the drug core with a permeable polymer, and encasing the coated drug core in an impermeable tube.

The present invention provides sustained-release formulations and devices for systemic or local delivery of nucleic acid-based therapeutic agents. In preferred embodiments, the subject invention provides methods and devices for treating or reducing the risk of viral infection, such as in the treatment of HIV, HPV, and CMV. Particularly preferred embodiments provide methods and devices for treating CMV retinitis by delivering to the eye a nucleic acid-based therapeutic agent that targets cytomegalovirus. The agent in such embodiments is preferably fomivirsen.

In other preferred embodiments, the invention provides methods and devices for inhibiting angiogenesis. Particularly preferred embodiments provide methods and devices for reducing angiogenesis within the eye, e.g., for treatment of age-related macular degeneration, by delivering to the eye a nucleic acid-based therapeutic agent that binds to VEGF. The agent in these embodiments is preferably pegaptanib.

In one embodiment, the invention relates to the use of antisense nucleic acid to decrease expression of a targeted disease-related protein. Such an antisense nucleic acid can be delivered, for example, as an expression plasmid which, when transcribed in the cell, produces RNA which is complementary to at least a unique portion of the cellular mRNA which encodes the targeted disease-related protein. Alternatively, the construct is an oligonucleotide which is generated ex vivo and which, when introduced into the cell causes inhibition of expression by hybridizing with the mRNA and/or genomic sequences encoding the targeted disease-related protein. Such oligonucleotides are optionally modified so as to be resistant to endogenous exonucleases and/or endonucleases. Exemplary nucleic acid molecules for use as antisense oligonucleotides are phosphoramidate, phosphothioate and methylphosphonate analogs of DNA (see for example U.S. Pat. Nos. 5,176,996; 5,264,564; and 5,256,775). General approaches to constructing oligomers useful in nucleic acid therapy have been reviewed, for example, by van der Krol et al., (1988) Biotechniques 6: 958-976; and Stein et al., (1988) Cancer Res 48: 2659-2668.

In other embodiments, the invention relates to the use of RNA interference (RNAi) to effect knockdown of the targeted gene. RNAi constructs comprise double stranded RNA that can specifically block expression of a target gene. RNAi constructs can comprise either long stretches of dsRNA identical or substantially identical to the target nucleic acid sequence, or short stretches of dsRNA identical or substantially identical to only a region of the target nucleic acid sequence.

Optionally, the RNAi constructs may contain a nucleotide sequence that hybridizes under physiologic conditions of the cell to the nucleotide sequence of at least a portion of the mRNA transcript for the gene to be inhibited (the “target” gene). The double-stranded RNA need only be sufficiently similar to natural RNA that it has the ability to induce RNAi. Thus, the invention contemplates embodiments that are tolerant of sequence variations that might be expected due to genetic mutation, polymorphic sites, or evolutionary divergence in a targeted sequence. The number of tolerated nucleotide mismatches between the target sequence and the RNAi construct sequence may be as high as 1 in 5 base pairs, but is preferably no higher than 1 in 10 base pairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Between 90% and 100% sequence identity between the inhibitory RNA and the portion of the target gene is preferred. Alternatively, the duplex region of the RNA may be defined functionally as a nucleotide sequence that is capable of detectably hybridizing with the target gene transcript after hybridization for 12 to 16 hours at 50° C. to 70° C. in 400 mM NaCl, 40 mM PIPES pH 6.4, and 1.0 mM EDTA, followed by washing.

The double-stranded structure may be formed by a single self-complementary RNA strand or two complementary RNA strands. Formation of the dsRNA may be initiated inside or outside of the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications.

The subject RNAi constructs can be “small interfering RNAs” or “siRNAs.” These nucleic acids are less than about 50, and preferably around 19-30 nucleotides in length, more preferably 21-23 nucleotides in length. The siRNAs are thought to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group. In certain embodiments, the siRNA constructs can be generated by processing of longer double-stranded RNAs, for example, in the presence of the enzyme DICER. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides. The siRNA molecules can be purified using a number of techniques known to those of skill in the art, such as gel electrophoresis. Alternatively, non-denaturing methods, such as column chromatography, size exclusion chromatography, glycerol gradient centrifugation, and affinity purification can be used to purify siRNAs.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis. Methods of chemically modifying RNA molecules can be adapted for modifying RNAi constructs (see, e.g., Heidenreich et al. (1997) Nucleic Acids Res. 25: 776-780; Wilson et al. (1994) J. Mol. Recog. 7: 89-98; Chen et al. (1995) Nucleic Acids Res. 23: 2661-2668; Hirschbein et al. (1997) Antisense Nucleic Acid Drug, Dev. 7: 55-61). For example, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted or 2′-deoxy ribonucleosides, α-configurations, etc.)

In some embodiments, at least one strand of the siRNA molecules may have a 3′ overhang from about 1 to about 6 nucleotides in length. Preferably, the 3′ overhangs are 1-3 nucleotides in length. In certain embodiments, one strand has a 3′ overhang and the other strand is blunt-ended or also has an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythymidine, may be tolerated without reducing the effectiveness of the RNAi. The absence of a 2′ hydroxyl significantly enhances the nuclease resistance of the overhang in tissue culture medium, and may be also beneficial in vivo.

The RNAi construct can also be in the form of a long double-stranded RNA, which is digested intracellularly to produce a siRNA sequence within the cell. Alternatively, the RNAi construct may be in the form of a hairpin RNA. It is known in the art that siRNAs can be produced by processing hairpin RNAs in the cell. Hairpin RNAs can be synthesized exogenously or can be formed by transcribing from RNA polymerase III promoters in vivo. Examples of making and using hairpin RNAs for gene silencing in mammalian cells are described in, for example, Paddison et al., Genes Dev, 2002, 16: 948-58; McCaffrey et al., Nature, 2002, 418: 38-9; McManus et al., RNA, 2002, 8: 842-50; Yu et al., Proc. Natl. Acad. Sci. USA, 2002, 99: 6047-52). Preferably, such hairpin RNAs are engineered in cells or in an animal to ensure continuous and stable suppression of a desired gene.

PCT application WO 01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

In another embodiment, the invention relates to the use of ribozyme molecules designed to catalytically cleave an mRNA transcript to prevent translation of the mRNA (see, e.g., PCT International Publication WO90/11364, published Oct. 4, 1990; Sarver et al., 1990, Science 247: 1222-1225; and U.S. Pat. No. 5,093,246). While any ribozyme that cleaves the target mRNA at a site-specific recognition sequence can be used to destroy that particular mRNA, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334: 585-591. The ribozymes of the present invention also include RNA endoribonucleases (“Cech-type ribozymes”) such as the one which occurs naturally in Tetrahymena thermophila (known as the IVS or L-19 IVS RNA) and which has been extensively described (see, e.g., Zaug, et al., 1984, Science, 224: 574-578; Zaug and Cech, 1986, Science, 231: 470-475; Zaug, et al., 1986, Nature, 324: 429-433; published International patent application No. WO88/04300 by University Patents Inc.; Been and Cech, 1986, Cell, 47: 207-216).

In a further embodiment, the invention relates to the use of DNA enzymes to inhibit expression of a targeted gene. DNA enzymes incorporate some of the mechanistic features of both antisense and ribozyme technologies. DNA enzymes are designed so that they recognize a particular target nucleic acid sequence, much like an antisense oligonucleotide; however, much like a ribozyme, they are catalytic and specifically cleave the target nucleic acid. Briefly, to design an ideal DNA enzyme that specifically recognizes and cleaves a target nucleic acid, one of skill in the art must first identify a unique (or nearly unique) target sequence. Preferably, the sequence is a G/C rich stretch of approximately 18 to 22 nucleotides. High G/C content helps insure a stronger interaction between the DNA enzyme and the target sequence. When synthesizing the DNA enzyme, the specific antisense recognition sequence that will target the enzyme to the message is divided so that it comprises the two arms of the DNA enzyme, and the DNA enzyme loop is placed between the two specific arms. Methods of making and administering DNA enzymes can be found, for example, in U.S. Pat. No. 6,110,462.

In certain embodiments, the nucleic acid-based therapeutic agents are prepared for sustained release from intraocular, intradermal, intramuscular, intraperitoneal, or subcutaneous sites. For instance, the nucleic acid-based therapeutic agent can be formulated in a polymer or hydrogel which can be introduced at site in the body where it remains reasonably dimensionally stable and localized for at least a period of days, and more preferably for 2-10 weeks or more. In other embodiments, the agent can be provided in a controlled- and sustained-release device, which in turn can be inserted at a position in the body, preferably where (by the location itself or by the use of means of securing the device) it is not likely to migrate from the compartment in which it is inserted.

One aspect of the invention provides a sustained release drug delivery device comprising an inner drug core comprising an amount of a nucleic acid-based therapeutic agent, and a dimensionally stable inner tube impermeable to the passage of said agent, the inner tube having first and second ends and covering at least a portion of the inner drug core. An impermeable member may be positioned at said inner tube first end, said impermeable member preventing passage of said agent out of said drug core through said inner tube first end, or alternatively a permeable member may be positioned at said inner tube first end, said permeable member allowing diffusion of said agent from said drug core through said inner tube first end. A permeable member is positioned at the inner tube second end, the permeable member allowing diffusion of the agent from the drug core through the inner tube second end.

Another aspect of the invention provides a sustained release drug delivery device comprising a drug core comprising an amount of a nucleic acid-based therapeutic agent, a first polymer coating permeable to the passage of said agent, and a second polymer coating impermeable to the passage of said agent, wherein the second polymer coating covers a portion of the surface area of the drug core and/or the first polymer coating.

Another aspect of the invention provides a sustained release drug delivery device comprising a drug core comprising an amount of an nucleic acid-based therapeutic agent, a first polymer coating and a second polymer coating permeable to the passage of said agent, wherein the two polymer coatings are bioerodable and erode at different rates.

A further aspect of the invention provides a sustained release drug delivery device comprising a drug core comprising an amount of a nucleic acid-based therapeutic agent, a first polymer coating permeable to the passage of said agent covering at least a portion of the drug core, a second polymer coating essentially impermeable to the passage of said agent covering at least a portion of the drug core and/or the first polymer coating, and a third polymer coating, permeable to the passage of said agent, covering the drug core and the second polymer coating, wherein a dose of said agent is released for at least 7 days.

Another aspect of the invention provides a sustained release drug delivery device comprising a drug core comprising an amount of a nucleic acid-based therapeutic agent, a first polymer coating permeable to the passage of said agent covering at least a portion of the drug core, a second polymer coating essentially impermeable to the passage of said agent covering at least a portion of the drug core and/or the first polymer coating, and a third polymer coating, permeable to the passage of said agent, covering the drug core and the second polymer coating, wherein release of said agent maintains a desired concentration of said agent for at least 7 days.

In the above-described embodiments, it is not necessary that the third polymer coating completely covers the drug core and second polymer coating. The third polymer coating may feature openings or ports which permit contact of biological fluids with the first and/or second polymer layers.

Yet still another aspect of the invention provides a sustained release drug delivery device comprising a drug core comprising an amount of a nucleic acid-based therapeutic agent, and a non-erodable polymer coating, the polymer coating being permeable to the passage of said agent covering the drug core and is essentially non-release rate limiting, wherein a dose of said agent is released for at least 7 days.

A further aspect of the invention provides a sustained release drug delivery device comprising a drug core comprising an amount of a nucleic acid-based therapeutic agent, and a non-erodable polymer coating, the polymer coating being permeable to the passage of said agent covering the drug core and being essentially non-release rate limiting, wherein release of said agent maintains a desired concentration of said agent for at least 7 days.

Yet another aspect of the invention provides a sustained release drug delivery device comprising a drug core comprising an amount of a nucleic acid-based therapeutic agent, a first polymer coating permeable to the passage of said agent covering at least a portion of the drug core, a second polymer coating impermeable to the passage of said agent covering at least 50% of the drug core and/or the first polymer coating, said second polymer coating comprising an impermeable film and at least one impermeable disc, and a third polymer coating permeable to the passage of said agent essentially completely covering the drug core, the uncoated portion of the first polymer coating, and the second polymer coating, wherein an dose of said agent is released for at least 7 days.

Another aspect of the invention provides a sustained release drug delivery device comprising a drug core comprising an amount of a nucleic acid-based therapeutic agent, a first polymer coating permeable to the passage of said agent covering at least a portion of the drug core, a second polymer coating impermeable to the passage of said agent covering at least 50% of the drug core and/or the first polymer coating, said second polymer coating comprising an impermeable film and at least one impermeable disc, and a third polymer coating permeable to the passage of said agent essentially completely covering the drug core, the uncoated portion of the first polymer coating, and the second polymer coating, wherein release of said agent maintains a desired concentration of said agent for at least 7 days.

In any of the above-described embodiments, the permeable coating may be bioerodable, and in such embodiments erosion of the permeable coating may occur concurrently with, or subsequent to, release of the nucleic acid-based therapeutic agent.

Yet still another aspect of the invention provides a method for treating age related macular degeneration and diabetic eye diseases, comprising inserting in the eye of a patient in need of such treatment a sustained release drug delivery device including a nucleic acid-based therapeutic agent that binds VEGF, wherein the dose of said agent is released for at least 7 days.

Yet still another aspect of the invention provides a method for treating age related macular degeneration and diabetic eye diseases, comprising inserting in the eye of a patient in need of such treatment a sustained release drug delivery device including a nucleic acid-based therapeutic agent that binds VEGF, wherein release of said agent maintains a desired concentration of said agent for at least 7 days.

As used herein, the expression “maintains a desired concentration” of an agent refers to the desired concentration of the agent at the intended site of action. For an agent intended to have systemic activity throughout the body, the desired concentration will typically be an effective concentration of the agent in the blood plasma, and in such cases it is the plasma concentration that is being referred to. Where the agent is intended to act locally, for example within the eye or within a body cavity, organ, or tumor, the desired concentration will be an effective concentration of agent within the eye or within that cavity, organ, or tumor, and in such cases it is the corresponding local concentration that is being referred to.

Codrugs or prodrugs may be used to deliver drugs, including the nucleic acid-based therapeutic agents of the present invention, in a sustained manner. In certain embodiments, codrugs and prodrugs may be adapted to use in the core or outer layers of the drug delivery devices described herein. An example of sustained-release systems using codrugs and prodrugs may be found in U.S. Pat. No. 6,051,576. This patent is incorporated in its entirety herein by reference. In other embodiments, codrugs and prodrugs may be included with the gelling, suspension, and other embodiments described herein.

As used herein, the term “constituent moiety” means one of two or more pharmaceutically active moieties so linked as to form a codrug according to the present invention as described herein. In some embodiments according to the present invention, two molecules of the same constituent moiety are combined to form a dimer (which may or may not have a plane of symmetry). In the context where the free, unconjugated form of the moiety is referred to, the term “constituent moiety” means a pharmaceutically active moiety, either before it is combined with another pharmaceutically active moiety to form a codrug, or after the codrug has been hydrolyzed to remove the linkage between the two or more constituent moieties. In such cases, the constituent moieties are chemically the same as the pharmaceutically active forms of the same moieties, or codrugs thereof, prior to conjugation.

As used herein, the term “codrug” means a first constituent moiety chemically linked to at least one other constituent moiety that is the same as, or different from, the first constituent moiety. The individual constituent moieties are reconstituted as the pharmaceutically active forms of the same moieties, or codrugs thereof, prior to conjugation. Constituent moieties may be linked together via reversible covalent bonds such as ester, amide, carbamate, carbonate, cyclic ketal, thioester, thioamide, thiocarbamate, thiocarbonate, xanthate and phosphate ester bonds, so that at the required site in the body they are cleaved to regenerate the active forms of the drug compounds.

The term “prodrug” is intended to encompass compounds that, under physiological conditions, are converted into the therapeutically active agents of the present invention. A common method for making a prodrug is to include selected moieties, such as esters, that are hydrolyzed under physiological conditions to convert the prodrug to an active biological moiety. In other embodiments, the prodrug is converted by an enzymatic activity of the host animal. Prodrugs are typically formed by chemical modification of a biologically active moiety. Conventional procedures for the selection and preparation of suitable prodrug derivatives are described, for example, in Design of Prodrugs, ed. H. Bundgaard, Elsevier, 1985.

In certain embodiments, the release of the nucleic acid-based therapeutic agent has a systemic effect. In other embodiments, the release of said agent has a local effect. The amount or dose of agent released from the drug delivery systems may be a therapeutically effective or a sub-therapeutically effective amount.

In some embodiments, the amount of the agent within the drug core or reservoir is at least 0.05 mg to about 500 mg, preferably at least about 0.5 mg, 30 mg, or 50 mg. In other embodiments, the amount of the agent within the drug core or reservoir is at least about 2 mg to about 15 mg, while in yet other embodiments it is about 15 mg to about 100 mg. In certain separate embodiments, a therapeutically effective amount or dose of the agent is released for at least two weeks, at least one month, at least two months, at least three months, at least 6 months, and at least one year.

In some embodiments, a therapeutically effective dose is at least about 30 ng/day, 30 ug/day, or 300 μg/day. In certain embodiments, the desired concentration of said agent in blood plasma is about 10-100 ng/ml, about 100-1000 ng/ml, or about 20-200 ug/ml.

In certain embodiments, the device is between about 1 to 30 mm in length, preferably about 3 mm, about 5 mm, about 7 mm, or about 10 mm. In certain embodiments, the device is between about 0.5 to 5 mm in diameter, preferably about 1 mm, about 2.5 mm, or about 4 mm.

In some embodiments, the permeable member comprises a material selected from cross-linked polyvinyl alcohol, poly(lactic acid) (PLA), poly(lactic-co-glycolic acid) (PLGA), poly(caprolactone) (PCL), polyolefins, polyvinyl chlorides, cross-linked gelatins, insoluble and non-erodable cellulose, acylated cellulose, esterified celluloses, cellulose acetate propionate, cellulose acetate butyrate, cellulose acetate phthalate, cellulose acetate diethylaminoacetate, polyurethanes, polycarbonates, and microporous polymers formed by co-precipitation of a polycation and a polyanion modified insoluble collagen. In preferred embodiments, the permeable member comprises cross-linked polyvinyl alcohol, PLA, PLGA, or PCL.

The permeable member may, in certain embodiments of the invention, incorporate positively-charged moieties, such as amino or quaternary ammonium groups, in order to modulate the rate of diffusion of the nucleic acid-based therapeutic agent from the device.

In certain embodiments, the impermeable member comprises a material selected from polyvinyl acetate, cross-linked polyvinyl butyrate, ethylene ethyl acrylate copolymer, polyethyl hexylacrylate, polyvinyl chloride, polyvinyl acetals, plasticized ethylene vinyl acetate copolymer, polyvinyl acetate, ethylene vinyl chloride copolymer, polyvinyl esters, polyvinyl butyrate, polyvinyl formal, polyamides, polyimide, nylon, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitrile copolymer. In preferred embodiments, the impermeable member comprises polyimide, silicone, PLA, PLGA, or PCL.

In some embodiments, the impermeable member is in the form of a tube.

In certain embodiments, the second polymer coating is a dimensionally stable tube. In some embodiments, the dimensionally stable tube includes one or more pores, for example, along the surface of the tube, to achieve the desired amount of drug released. The shape of a pore is not limited to any particular shape but may be in the shape of a slit, a circular hole, or any other geometrical shape.

In some embodiments, the drug core comprises a pharmaceutically acceptable carrier. In certain embodiments, the drug core comprises 0.1 to 100% drug. In one embodiment, the drug core comprises 0.1 to 100% drug, 0.1 to 10% magnesium stearate, and 0.1 to 10% polyethylene glycol. The drug core may also, additionally or alternatively, comprise one or more positively charged carriers. Positively charged carriers include charged polymers, preferably polycationic polymers, such as chitosan, polyethyleneimine, DEAE dextran, poly lysine, poly(Lys₅-Cys-SS-Cys-Lys₅), and the like, which bind the nucleic acid-based therapeutic agent and modulate the rate of release. Use of charged polymers for this purpose is known in the art, as described for example in U.S. Pat. No. 6,645,525 and in M. L. Read et al., J. Gene Med. 2003, 5: 232-245. Charged carriers suitable for use in the invention also include but are not limited to biogenic polyamines, such as spermine, spermidine and putrescine, and cationic amphiphilies such as DOTAP (1,2-bis(oleoyloxy)-3-trimethylammonium-propane), DOTMA (N-[1-(2,3-dioleoyloxy)propyl]-N,N,N-trimethyl ammonium chloride), DDAB (dimethyldioctadecylammonium bromide), DC-cholesterol (3-β-[N-(N′,N′-dimethylaminoethane)carbamoyl]cholesterol), and DODAP (1,2-bis(oleoyloxy)-3-dimethylammoniumpropane). The use of positively-charged lipid carriers to improve the efficiency of cell transfection with DNA or RNA is well-established; see for example U.S. Pat. No. 6,670,393 and references therein. The charged carriers may also be incorporated into the permeable layers or members of the devices described herein.

Any pharmaceutically acceptable form of a nucleic acid-based therapeutic agent may be employed in the practice of the present invention. Pharmaceutically acceptable salts, for instance, include sodium, potassium, magnesium, and calcium salts, as well as sulfate, lactate, acetate, stearate, hydrochloride, tartrate, maleate, and the like.

The drug delivery device of the present invention may be administered to a mammalian organism via any route of administration known in the art. Such routes of administration include intraocular, oral, subcutaneous, intramuscular, intraperitoneal, intranasal, dermal, into the brain, including intracranial and intradural, into the joints, including ankles, knees, hips, shoulders, elbows, wrists, directly into tumors, and the like. In addition, one or more of the devices may be administered at one time, or more than one agent may be included in the inner core or reservoir, or more than one reservoir may be provided in a single device.

For systemic relief, the devices may be inserted subcutaneously, intramuscularly, intraarterially, intrathecally, or intraperitoneally. This is the case when devices are to give sustained systemic levels and avoid premature metabolism. In addition, such devices may be administered orally.

For localized drug delivery, the devices may be surgically implanted at or near the desired site of action. This may be the case for devices of the present invention used in treating ocular conditions, primary tumors, rheumatic and arthritic conditions, and chronic pain.

In certain embodiments, a device and method of preparation thereof that is suitable for the controlled and sustained release of a nucleic acid-based therapeutic agent includes sealing at least one surface of a reservoir of the device with an impermeable member which is capable of supporting its own weight, which has dimensional stability, which has the ability to accept a drug core therein without changing shape, and/or retains its own structural integrity so that the surface area for diffusion does not significantly change, manufacture of the entire device is made simpler and the device is better able to deliver the agent.

The use of a tube of material to hold the drug reservoir during manufacture allows for significantly easier handling of the tube and reservoir, because the tube fully supports both its own weight and the weight of the reservoir. Thus, the tube used in the present invention is not a coating, because a coating cannot support its own weight. Also, this rigid structure allows the use of drug slurries drawn into the tube, which allows the fabrication of longer cylindrical devices. Furthermore, because of the relative ease of manufacturing such devices, more than one reservoir, optionally containing more than one drug, can be incorporated into a single device.

During use of the devices, because the size, shape, or both, of the drug reservoir typically changes as drug diffuses out of the device, the tube which holds the drug reservoir is sufficiently strong or rigid to maintain a diffusion area so that the diffusion rate from the device does not change substantially because of the change in size or surface area of the drug reservoir. By way of example and not of limitation, an exemplary method of ascertaining if the tube is sufficiently rigid is to form a device in accordance with the present invention, and to measure the diffusion rate of the drug from the device over time. If the diffusion rate changes more than 50% from the diffusion rate expected based on the chemical potential gradient across the device at any particular time, the tube has changed shape and is not sufficiently rigid. Another exemplary test is to visually inspect the device as the drug diffuses over time, looking for signs that the tube has collapsed in part or in full.

The use of permeable and impermeable tubes in accordance with the present invention provides flow resistance to reverse flow, i.e., flow back into the device. The tube or tubes assist in preventing large proteins from solubilizing the drug in the drug reservoir. Also, the tube or tubes assist in preventing oxidation and protein lysis, and limit the entry of other biological agents that might degrade or erode the contents of the reservoir.

The present invention contemplates a device and method for delivering and maintaining a therapeutic amount of at least one nucleic acid-based therapeutic agent in the eye of a patient for an extended period of time. The device is a sustained-release drug delivery device comprising at least one nucleic acid-based therapeutic agent, which can maintain a therapeutically effective concentration of the nucleic acid-based therapeutic agent within the eye for an extended period of time. The method involves inserting such a device into or in proximity to the eye of a patient, so as to deliver the nucleic acid-based therapeutic agent to the retina.

The device of the present invention may be adapted for insertion between the eye and eyelid, preferably the lower eyelid. It may, in preferred embodiments, be adapted for insertion into the anterior or posterior chambers, under the retina, into the choroid, or into or onto the sclera. In another embodiment, the device may be adapted for insertion into the lacrimal canaliculus. In yet another embodiment, the device may be a contact lens or intraocular lens, or it may be incorporated into or attached to a contact lens or intraocular lens.

As used herein, “sustained-release device” or “sustained-release formulation” means a device or formulation that releases an agent over an extended period of time in a controlled fashion. As also discussed elsewhere herein, examples of sustained-release devices and formulations suitable for the present invention may be found in U.S. Pat. No. 6,375,972, U.S. Pat. No. 5,378,475, U.S. Pat. No. 5,773,019, and U.S. Pat. No. 5,902,598. The disclosures of these patents are incorporated herein by reference.

In one embodiment, the present invention provides a sustained-release drug delivery device adapted for insertion into or adjacent to the eye of a patient, where the drug delivery device, in whole or in part, is formed by co-extruding (a) an inner drug-containing core comprising at least one nucleic acid-based therapeutic agent and (b) an outer polymeric layer. The outer layer, which preferably is tubular in shape, may be permeable, semi-permeable, or impermeable to the agent. In certain embodiments, the agent-containing core may be formed by admixing the agent with a polymer matrix prior to formation of the device. In such case, the polymer matrix may or may not significantly affect the release rate of the agent. The outer layer, the polymer admixed with the drug-containing core, or both may be bioerodable. The co-extruded product can be segmented into a plurality of drug delivery devices. The devices may be left uncoated so that their respective ends are open, or the devices may be coated partially or completely with, for example, an additional polymeric layer that is permeable, semi-permeable, or impermeable to the agent. Alternatively, the core may be extruded and the polymeric layer added by methods such as dip coating, film coating, spray coating, and the like.

As more fully described in copending U.S. patent application Ser. No. 10/428,214, filed May 2, 2003, and Ser. No. 10/714,549, filed Nov. 13, 2003, and in U.S. Provisional Patent Application 60/501,947, filed Sep. 11, 2003, the disclosures of each of which are incorporated by reference herein in their entirety, the co-extruded embodiment discussed above may be fabricated by forwarding a polymeric material to a first extrusion device, forwarding at least one drug to a second extrusion device, co-extruding a mass including the polymeric material and the drug, and forming the mass into at least one co-extruded drug delivery device that comprises a core including the drug(s) and an outer layer including the polymeric material. In certain embodiments, the drug(s) forwarded to the second extrusion device is in admixture with at least one polymer. The polymer(s) may be a bioerodable polymer, such as poly(vinyl acetate) (PVAC), polycaprolactone (PCL), polyethylene glycol (PEG), or poly(dl-lactide-co-glycolide) (PLGA). In certain embodiments, the drug(s) and the at least one polymer are admixed in powder form.

The outer layer may be impermeable, semi-permeable, or permeable to the drug disposed within the inner drug-containing core, and may comprise any biocompatible polymer, such as PCL, an ethylene/vinyl acetate copolymer (EVA), polyalkyl cyanoacrylate, polyurethane, a nylon, or PLGA, or a copolymer of any of these. In certain embodiments, the outer layer is radiation curable. In certain embodiments, the outer layer comprises at least one drug, which may be the same or different than the drug used in the inner core.

While co-extrusion may be used to form a device according to the invention, other techniques may readily be used. For example, the core can be poured or injected into a preformed tube otherwise having one or more of the characteristics of the present invention. In certain embodiments, the drug delivery device (formed by any of the possible techniques) is in a tubular form, and may be segmented into a plurality of shorter products. In certain embodiments, the plurality of shorter products may be coated partially or completely with one or more additional layers, including at least one of a layer that is permeable to the nucleic acid-based therapeutic agent, a layer that is semi-permeable to such drug(s), and a layer that is bioerodable. The additional layer(s) may include any biocompatible polymer, such as PCL, EVA, polyalkyl cyanoacrylate, polyurethane, a nylon, or PLGA, or a copolymer of any of these.

Materials suitable to form the outer layer and inner drug-containing core, respectively, are numerous. In this regard, U.S. Pat. No. 6,375,972, the disclosures of which are incorporated herein by reference, describes suitable materials for forming insertable co-extruded drug delivery devices, which materials are included among those usable as materials for the outer layer and inner drug-containing core. Preferably, the materials for certain embodiments of the present invention are selected for their ability to be extruded without negatively affecting the properties for which they are specified. For example, for those materials that are to be impermeable to the drug, a material is selected that, upon being processed through an extrusion device, is or remains impermeable. Similarly, biocompatible materials are preferably chosen for the materials that will, when the drug delivery device is fully constructed, come in contact with the patient's biological tissues. Suitable materials include PCL, EVA, PEG, poly(vinyl acetate) (PVA), poly(lactic acid) (PLA), poly(glycolic acid) (PGA), PLGA, polyalkyl cyanoacrylate, polyurethane, nylons, or copolymers thereof. In polymers including lactic acid monomers, the lactic acid may be D-, L-, or any mixture of D- and L-isomers.

The selection of the material(s) to form the inner drug-containing core involves additional considerations. As one of skill in the art readily appreciates, extrusion devices typically include one or more heaters and one or more screw drives, plungers, or other pressure-generating devices; indeed, it may be a goal of the extruder to raise the temperature, fluid pressure, or both, of the material being extruded. This can present difficulties when a pharmaceutically active drug included in the materials being processed and extruded by the extruder is heated and/or exposed to elevated pressures. This difficulty can be compounded when the drug itself is to be held in a polymer matrix, and therefore a polymer material is also mixed and heated and/or pressurized with the drug in the extruder. The materials may be selected so that the activity of the drug in the inner drug-containing core is sufficient for producing the desired effect when inserted in a patient. Furthermore, when the drug is admixed with a polymer for forming a matrix upon extrusion, the polymer material that forms the matrix is advantageously selected so that the drug is not destabilized by the matrix. Preferably, the matrix material is selected so that diffusion through the matrix has little or no effect on the release rate of the nucleic acid-based therapeutic agent from the matrix.

The materials from which the product is made may be selected to be stable during the release period for the drug delivery device. The materials may optionally be selected so that, after the drug delivery device has released the nucleic acid-based therapeutic agent for a predetermined amount of time, the drug delivery device erodes in situ, i.e., is bioerodable. The materials may also be selected so that, for the desired life of the delivery device, the materials are stable and do not significantly erode, and the pore size of the materials does not change. In certain embodiments using a matrix with the drug core, the matrix is bioerodable, while in other embodiments the matrix is non-bioerodable.

There are at least two functions of matrix material selected for the inner drug-containing core: to permit ease of manufacture of the core, whether by compression, extrusion, co-extrusion or some other process; and to inhibit, or prevent, decomposition of the drug in the core due to the migration into the matrix of biological molecules. The matrix material of the inner drug-containing core inhibits, and preferably prevents, the passage of enzymes, proteins, and other materials into the drug-containing core that would lyse the drug before it has an opportunity to be released from the device. As the core empties, the matrix may weaken and break down. Then, the outer layer will be exposed to degradation from both the outside and inside from water and enzymatic action. Drugs having higher solubilities are preferably linked to form low solubility conjugates; alternatively, drugs may be linked together to form molecules large enough or sufficiently insoluble to be retained in the matrix.

In addition to one or more nucleic acid-based therapeutic agents and matrix-forming polymers, the inner agent-containing core may include positively-charged carriers as described above, as well as materials such as lipids (including long chain fatty acids) and waxes, anti-oxidants, and in some cases, release modifiers (e.g., water or surfactants). These materials should be biocompatible and remain stable during the manufacturing process. In certain embodiments, the blend of active agent, polymers, and other materials should be extrudable under desired processing conditions. The matrix-forming polymers or any materials used should be able to carry a sufficient amount of active agent to produce therapeutically effective actions over the desired period of time. It is also preferred that the materials used as carriers have no deleterious effect on the activity of the nucleic acid-based therapeutic agent.

In certain embodiments, the matrix polymer(s) may be selected so that the release rate of the agent from the matrix is determined, at least in part, by the physico-chemical properties of the agent, and not by the properties of the matrix. Alternatively, the matrix may be selected such that it modifies the release rate of the agent. For example, where a nucleic acid-based agent is in polyanionic form, the matrix may include protonated basic moieties having a pKa that is higher than that of the agent, or quaternary nitrogen moieties, which electostatically bind to the polyanions, thereby slowing the release rate of the agent. Where a nucleic acid-based agent is in neutral (protonated) form, the matrix may have acidic moieties having a pKa that is relatively close to that of the agent, wherein the matrix functions as a buffer to the deprotonation of the polynucleotide and thereby slows its release from the device. In addition, the pH microenvironment of the matrix may be varied by the addition of acids or by the use of phosphate or other buffers, thereby controlling the protonation state of the agent and its rate of diffusion from the matrix. In certain embodiments, the matrix material is selected so that the sustained release rate of the agent is controlled by the rate of its deprotonation, so that the agent's diffusion rate through the matrix has little or no effect on the agent's release rate from the matrix.

In certain embodiments, the agent may also be included in the outer layer. This may provide biphasic release with an initial burst such that when such a system is first placed in the body, a substantial fraction of the total agent released is released from the outer layer. Subsequently, more agent is released from the inner agent-containing core. The agent included in the outer layer may be different from the agent included in the core.

As noted in certain examples of the co-extruded embodiment described herein, it will be appreciated that a variety of materials may be used for the outer layer to achieve different release rate profiles. For example, as discussed in the aforementioned '972 patent, an outer layer may be surrounded by a permeable or impermeable additional layer, or may itself be formed of a permeable or semi-permeable material. Accordingly, co-extruded devices of the present invention may be provided with one or more outer layers using techniques and materials fully described in the '972 patent. Through the use of permeable or semi-permeable materials, drug(s) in the core may be released at various rates. In addition, even materials considered to be impermeable may permit release of drug(s) or other active agents in the core under certain circumstances. Thus, permeability of the outer layer may contribute to the release rate of an agent over time, and may be used as a parameter to control the release rate over time for a deployed device.

In certain embodiments, the agent has a permeability coefficient in the outer layer of less than about 1×10⁻¹⁰ cm/s. In other embodiments the permeability coefficient in the outer layer is greater than 1×10⁻¹⁰ cm/s, or even greater than 1×10⁻⁷ cm/s. In certain embodiments the permeability coefficient is at least 1×10⁻⁵ cm/s, or even at least 1×10⁻³ cm/s, or at least 1×10⁻² cm/s.

Further, devices may be segmented into devices having, for example, an impermeable outer layer surrounding an inner agent-containing core, with each segment being optionally further coated by a semi-permeable or permeable layer to control a release rate through the exposed ends thereof. Similarly, the outer layer, or one or more additional layers surrounding the device, may be bioerodable at a known rate, so that core material is exposed after a certain period of time along some or all of the length of the tube, or at one or both ends thereof. Thus, it will be appreciated that, using various materials for the outer layer and one or more additional layers surrounding a co-extruded device, the delivery rate for the deployed device may be controlled to achieve a variety of release rate profiles.

As more fully described in U.S. Provisional Application No. 60/483,316, the disclosure of which is incorporated herein by reference, certain embodiments provide a polymer drug delivery system (“polymer system”) comprising an inner core or reservoir (“inner core”) that contains a therapeutically effective amount of an agent, a first coating layer that is impermeable, negligibly or partially permeable to the agent and, optionally, a second coating layer that is permeable or semi-permeable to the agent. Additional layers may also optionally be used.

In certain embodiments, the inner agent-containing core has biocompatible fluid and biocompatible solid components, where the biocompatible solid is less soluble in physiological fluid than in the biocompatible fluid. The biocompatible fluid may be hydrophilic, hydrophobic or amphiphilic; may be polymeric or nonpolymeric. Such fluid may also be a biocompatible oil. In certain embodiments, a biocompatible solid (e.g., a bioerodable polymer) is dissolved, suspended, or dispersed in the biocompatible fluid (to form a “biocompatible core component”). At least one agent, such as a nucleic acid-based therapeutic agent, is also dispersed, suspended, or dissolved in the biocompatible core component.

The first coating layer surrounds the inner core, is an impermeable, negligibly or partially permeable polymer, and may feature one or more diffusion ports or pores (“ports”) that further allow the agent to diffuse from the core out of the system. The rate of agent release from such systems may be controlled by the permeability of a matrix in the inner core (as described below), the solubility of the agent in the biocompatible core component, the thermodynamic activity of the agent in the biocompatible core component, the potential gradient of the agent from the inner core to the biological fluid, the size of the diffusion port(s), and/or the permeability of the first or second coating layer.

The first coating layer includes at least one polymer and is preferably bioerodible, but it may alternatively be non-bioerodible. The first coating layer covers at least part but preferably not all of the surface of the inner core, leaving at least one opening as a diffusion port through which the agent can diffuse. If a second coating layer is used, it may partially cover or cover essentially all of the first coating layer and inner core, and its permeability to the agent permits the agent to diffuse into the surrounding fluid.

The first coating, in addition to or as an alternative to providing one or more diffusion ports, may further comprise a permeability-modifying component that erodes in vivo, or it may comprise two or more different polymers (e.g., having different monomer units, different molecular weights, different degrees of crosslinking, and/or different molar ratios of monomer units), at least one of which is a permeability-modifying component that erodes in vivo, such that after implantation the first coating itself is capable of becoming permeable to the active agent. Permeability-modifiying components include but are not limited to water-soluble polymers. A preferred permeability-modifying component that erodes in vivo is polyethylene glycol. For example, modifying a poly-(D,L-lactide-co-glycolide)(PLGA) coating by adding 20% polyethylene glycol to the polymer, and coating a drug core containing 1:1 albumin-PLGA with the modified polymer, results in a device that begins to release albumin several days earlier than an identical device coated with unmodified PLGA.

A variety of materials may be suitable to form the coating layer(s) of these embodiments of the present invention. Preferable polymers are largely insoluble in physiological fluids. Suitable polymers may include naturally occurring or synthetic polymers. Certain exemplary polymers include, but are not limited to, PVA, cross-linked polyvinyl alcohol, cross-linked polyvinyl butyrate, ethylene ethylacrylate copolymer, polyehtyl hexylacrylate, polyvinyl chloride, polyvinyl acetals, plasticized ethylene vinylacetate copolymer, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene chlorinated polyethylene, poly(1,4-isopropylidene dipehenylene carbonate), vinylidene chloride, acrylonitrile copolymer, vinyl-chloride-diethyl fumarate copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer, and vinylidene chloride-acrylonitrile copolymer.

As noted above, where applied, the biocompatible core component includes at least one biocompatible solid (e.g., a bioerodable polymer) that is at least partially dissolved, suspended, or dispersed in a biocompatible polymeric or nonpolymeric fluid or a biocompatible oil. Further, the biocompatible solid is more soluble in the biocompatible fluid or oil than the physiological fluid such that, when the device is placed in contact with physiological fluid, the biocompatible core component precipitates or undergoes a phase transition. The inner core may be delivered as a gel. It may preferably be delivered as a particulate or a liquid that converts to a gel upon contact with water or physiological fluid. In some embodiments, the nonpolymeric fluid may include the agent in acidic form.

In certain embodiments, the biocompatible fluid of the biocompatible core component is hydrophilic (e.g., PEG, cremophor, polypropylene glycol, glycerol monooleate, and the like), hydrophobic, or amphiphilic. In certain embodiments, said fluid may be a monomer, polymer or a mixture of the same. If used, the biocompatible oil may be sesame oil, miglyol, or the like.

In certain embodiments, injectable liquids may be used that, upon injection, undergo a phase transition and are transformed in situ into gel delivery vehicles. In certain embodiments, at least one polymer in the inner core may convert from an agent-containing liquid phase to an agent-infused gel phase upon exposure to a physiological fluid. Technologies based on in situ gelling compositions are described in U.S. Pat. Nos. 4,938,763, 5,077,049, 5,278,202, 5,324,519, and 5,780,044, all of which may be adapted to such embodiments of the present invention, and the disclosure of each of which is incorporated herein by reference. In certain embodiments, the biocompatible solid of the biocompatible core component may be, for example, but without limitation, PLGA. In certain embodiments, the inner core is a viscous paste containing at least 10% agent, or preferably over 50% agent or, more preferably, over 75% agent.

In certain embodiments, the inner core comprises an in situ gelling drug delivery formulation comprising: (a) one or more nucleic acid-based therapeutic agents; (b) a liquid, semi-solid, or wax PEG; and (c) a biocompatible and bioerodable polymer that is dissolved, dispersed, or suspended in the PEG. The formulation may optionally also contain additives, such as pore-forming agents (e.g., sugars, salts, and water-soluble polymers), positively-charged carriers as described above, and release rate modifiers (e.g., sterols, fatty acids, glycerol esters, and the like). As more fully described in U.S. Provisional Patent Application No. 60/482,677, the disclosure of which is incorporated herein in its entirety, such formulation, on contact with water or bodily fluids, undergoes exchange of the PEG for water, resulting in precipitation of both the polymer and the agent and subsequent formation of a gel phase within which the agent is incorporated. The agent subsequently diffuses from the gel over an extended period of time.

A “liquid” PEG is a polyethylene glycol that is a liquid at 20-30° C. and ambient pressure. In certain preferred embodiments, the average molecular weight of the liquid PEG is between about 200 and about 400 amu. The PEG may be linear or it may be a bioabsorbable branched PEG, for example as disclosed in U.S. Patent Application No. 2002/0032298. In certain alternative embodiments, the PEG may be a semi-solid or wax, in which case the molecular weight will be larger, for example 3,000 to 6,000 amu. It will be understood that compositions comprising semi-solid and waxy PEGs may not be amenable to injection, and will accordingly be inserted by alternative means.

In certain embodiments, the nucleic acid-based therapeutic agent is dissolved in PEG, while in other embodiments, the agent is dispersed or suspended in PEG in the form of solid particles. In yet other embodiments, the agent may be encapsulated or otherwise incorporated into particles, such as microspheres, nanospheres, liposomes, lipospheres, micelles, and the like, or it may be conjugated to a polymeric carrier. Any such particles are preferably less than about 500 microns in diameter, more preferably less than about 150 microns.

The polymer that is dissolved, dispersed, or suspended in PEG of the formulation discussed above may be any biocompatible PLGA polymer that is soluble in or miscible with PEG, and is less soluble in water. It is preferably water-insoluble, and is preferably a bioerodable polymer. The carboxyl termini of the lactide- and glycolide-containing polymer may optionally be capped, e.g., by esterification, and the hydroxyl termini may optionally be capped, e.g., by etherification or esterification. Preferably, the polymer is PLGA having a lactide:glycolide molar ratio of between 20:80 and 90:10, more preferably between 50:50 and 85:15.

The term “bioerodable” is synonymous with “biodegradable” and is art-recognized. It includes polymers, compositions and formulations, such as those described herein, that degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, biodegradation may occur by enzymatic mediation, degradation in the presence of water and/or other chemical species in the body, or both.

The terms “biocompatible” and “biocompatibility” when used herein are art-recognized and mean that the referent is neither itself toxic to a host (e.g., an animal or human), nor degrades (if it degrades) at a rate that produces byproducts (e.g., monomeric or oligomeric subunits or other byproducts) at toxic concentrations, causes inflammation or irritation, or induces an immune reaction, in the host. It is not necessary that any subject composition have a purity of 100% to be deemed biocompatible. Hence, a subject composition may comprise 99%, 98%, 97%, 96%, 95%, 90% 85%, 80%, 75% or even less of biocompatible agents, e.g., including polymers and other materials and excipients described herein, and still be biocompatible.

In certain embodiments, a polymer system is injected or otherwise inserted into a physiological system (e.g., a patient). Upon injection or other insertion, the polymer system will contact water or other immediately surrounding physiological fluid that will enter the polymer system and contact the inner core. In certain embodiments, the core materials may be selected so as to create a matrix that reduces (and thereby allows control of) the rate of release of the agent from the polymer system.

In preferred embodiments, the agent's rate of release from the polymer system is limited primarily by the permeability or solubility of the agent in the matrix. However, the release rate may be controlled by various other properties or factors. For example, but without limitation, the release rate may be controlled by the size of the diffusion port(s), the permeability of the second coating layer of the polymer system, the physical properties of the inner core, the dissolution rate of the inner core or components of said core, or the solubility of the agent in the physiological fluid immediately surrounding the polymer system.

In certain embodiments, the rate of release of the agent may be limited primarily by any of the foregoing properties. For example, in certain embodiments, the rate of release of the agent may be controlled, or even limited primarily by, the size of the diffusion port(s). Depending on the desired delivery rate of the agent, the first coating layer may coat only a small portion of the surface area of the inner core for faster release rates of the agent (i.e., the diffusion port(s) is relatively large), or may coat large portions of the surface area of the inner core for slower release rates of the agent (i.e., the diffusion port(s) is relatively small).

For faster release rates, the first coating layer may coat up to about 10% of the surface area of the inner core. In certain embodiments, approximately 5-10% of the surface area of the inner core is coated with the first coating layer for faster release rates.

Certain embodiments may achieve desirable sustained release if the first coating layer covers at least 25% of the surface area of the inner core, preferably at least 50% of the surface area, more preferably at least 75%, or even greater than 85% or 95% of the surface area. In certain embodiments, particularly where the agent is readily soluble in both the biocompatible core component and the biological fluid, optimal sustained release may be achieved if the first coating layer covers at least 98% or 99% of the inner core. Thus, any portion of the surface area of the inner core, up to but not including 100%, may be coated with the first coating layer to achieve the desired rate of release of the agent.

The first coating layer may be positioned anywhere on the inner core, including, but not limited to, the top, bottom, or any side of the inner core. In addition, it could be positioned on the top and a side, or the bottom and a side, or the top and the bottom, or on opposite sides or on any combination of the top, bottom, or sides. As described herein, it may also cover the inner core on all sides while leaving a relatively small uncovered place as a port. In preferred embodiments, the inner core is cylindrical in shape, and the first coating layer covers the sides of the cylinder while leaving the ends of the cylinder uncoated. Such embodiments are produced, for example, by segmentation of a coated, continuously extruded or co-extruded core. Permeable caps or plugs, or a permeable second coating layer, preferably cover one end, or more preferably cover both ends, of these embodiments.

The composition of the first coating layer is selected so as to allow the above-described controlled release. The preferred composition of the first layer may vary depending on such factors as the active agent, the desired rate of release of the agent and the mode of administration. The identity of the active agent is important because its molecular size may determine, at least in part, its rate of release into the second coating layer if used.

In certain of such embodiments, the release rate of the agent from the inner core may be reduced by the permeability of the second coating layer. In certain embodiments, the second coating layer is freely permeable to the agent. In certain embodiments, the second coating layer is semi-permeable to the agent. In certain embodiments, the agent has a permeability coefficient in the second coating layer of less than about 1×10⁻¹⁰ cm/s. In other embodiments the permeability coefficient in the second coating layer is greater than 1×10⁻¹⁰ cm/s, or even greater than 1×10⁻⁷ cm/s. In certain embodiments the permeability coefficient is at least 1×10⁻⁵ cm/s, or even at least 1×10⁻³ cm/s, or at least 1×10⁻² cm/s in the second layer.

In certain embodiments, the inner core undergoes a phase change and converts to a gel upon insertion of the polymer system in a physiological system. The phase change may reduce the rate of release of the agent from the inner core. For example, where at least part of the inner core is provided first as a liquid and converts to a gel, the gel phase of the biocompatible core component may be less permeable to the agent than is the liquid phase. In certain embodiments, the biocompatible core component in gel phase is at least 10% or even at least 25% less permeable to the agent than is the liquid phase. In other embodiments, the precipitated biocompatible solid is at least 50% or even at least 75% less permeable to the agent than is the biocompatible fluid. In certain embodiments, interaction of the inner core with the physiological fluid may alter the solubility of the agent in the core. For example, the inner core is at least 10% or even at least 25% less solubilizing to the agent than before interaction with physiological fluid. In other embodiments, the gel phase is at least 50% or even at least 75% less solubilizing.

In certain embodiments, the rate at which the biocompatible solid and/or fluid components of the inner core dissolve may impact the rate of release of the agent. In certain embodiments, as the biocompatible core component erodes or dissolves, the rate of release of the agent may increase. For example, less than about 10% of the biocompatible core component may erode over a period of about 6 hours. This may increase the rate of release of the agent by less than about 10% over that time. In certain embodiments, the biocompatible core component may erode or dissolve more slowly (e.g., less than about 10% over a period of about 24 hours, or even over a period of multiple days, weeks, or even months). In certain embodiments, such erosion may occur more rapidly (e.g., greater than about 10% over a period of about 6 hours, in certain embodiments even greater than 25% over a period of about 6 hours).

In certain embodiments, the release rate of the agent from the inner core may be controlled by the ratio of the agent to the biocompatible solid component of the core (also referred to as the “drug loading”). By changing the drug loading, different release rate profiles can be obtained. Increasing the drug loading may increase the release rate. For a slower release profile, drug loading may be less than 10%, and preferably less than 5%. For a faster release profile, drug loading may be more than 10%, and preferably more than 20%, or even greater than 50%.

Thus, the rate of release of the agent according to the invention may be limited primarily by any of the above properties or any other factor. For example, but without limitation, the release rate may be controlled by the size and/or location of the diffusion port(s), the permeability or other properties of the first or a second coating layer in the polymer system, the physical properties of the inner core, the dissolution rate of the biocompatible core component, the solubility of the agent within the inner core, the solubility of the agent in the physiological fluid immediately surrounding the polymer system, etc.

In certain embodiments, the coating layer(s) may be formed with the nucleic acid-based therapeutic agent as a substantially homogeneous system, formed by mixing one or more suitable monomers with the agent, then polymerizing the monomer to form a polymer system. In this way, the agent is dissolved or dispersed in the polymer. In other embodiments, the agent is mixed into a liquid polymer or polymer dispersion and then the polymer is further processed to form the inventive coating(s). Suitable further processing may include crosslinking with suitable crosslinking agents, further polymerization of the liquid polymer or polymer dispersion, copolymerization with a suitable monomer, block copolymerization with suitable polymer blocks, etc. The further processing traps the agent in the polymer so that the agent is suspended or dispersed in the polymer system.

Another embodiment of the present invention provides a sustained-release drug delivery device adapted for insertion into or adjacent to the eye of a patient, where the drug delivery device comprises:
(i) an inner drug core comprising at least one nucleic acid-based therapeutic agent;
(ii) a first coating that is impermeable to the passage of the at least one nucleic acid-based therapeutic agent, having one or more openings therein through which the at least one nucleic acid-based therapeutic agent can diffuse, and which is substantially insoluble and inert in body fluids and compatible with body tissues; and
(iii) one or more additional coatings that are permeable to the passage of the at least one nucleic acid-based therapeutic agent, and which are substantially insoluble and inert in body fluids and compatible with body tissues;
wherein the impermeable and permeable coatings are disposed about the inner core so as to produce, when inserted, a constant rate of release of the at least one nucleic acid-based therapeutic agent from the device. Such a sustained-release device is disclosed in U.S. Pat. No. 5,378,475.

While embodiments of the device described in the '475 patent solve many of the problems pertaining to drug delivery, polymers suitable for coating the inner core are frequently relatively soft and technical difficulties can arise in the production of uniform films. This is especially true when attempting to coat non-spherical bodies with edges, such as those having a cylindrical shape. In such cases, relatively thick films must be applied to achieve uninterrupted and uniform coatings, which adds significant bulk to the device. Alternatively, the added bulk of the film coating can be accommodated by limiting the internal volume of the device, but this limits the amount of drug that can be delivered, potentially limiting both efficacy and duration.

The issue of device size is extremely important in the design of devices for insertion into or in the vicinity of the eye. Larger devices require more complex procedures to both insert and remove, and involve an associated increased risk of complications, longer healing or recovery periods, and potential side effects.

The aforementioned U.S. Pat. No. 5,902,598 presents solutions to the problems of manufacturing devices that are small enough for insertion into or in the vicinity of the eye, by loading a drug composition into a preformed shell rather than attempting to coat the drug core, but manufacturing difficulties can arise with this method. In particular, the impermeable inner coating layer that immediately surrounds the drug reservoir is typically so thin that the shell is not capable of supporting its own weight. While beneficial from the standpoint of reducing the size of the device while still sealing the drug reservoir, the relative flaccidity of this inner layer makes it difficult to load the reservoir with a drug. Because this inner layer does not have the dimensional stability or structural strength to accept the introduction of a drug core without changing shape, a relatively solid drug or drug-containing mixture must be used in order to manufacture the device. Loading a drug slurry into an inner layer that does not hold its own shape results in the combination of the drug slurry and inner layer being extremely difficult to handle during manufacture without damaging it, because the inner layer collapses and the drug-containing mixture flows out. An illustrative analogy may be made to the task of filling a plastic bag with water.

As more fully described in U.S. Pat. No. 6,375,972, the disclosure of which is incorporated by reference herein, yet another embodiment of the present invention addresses these problems by providing a sustained-release drug delivery system comprising an inner reservoir containing a drug core comprising at least one nucleic acid-based therapeutic agent, and an inner tubular covering that is impermeable to the passage of the agent and that covers at least a portion of the drug core. It will be appreciated that the invention operates on the premise that diffusion through the permeable layer(s) is faster than diffusion through the impermeable layer.

The inner tubular covering is sized and formed of a material so that it is capable of supporting its own weight, and has first and second ends such that the tubular covering and the two ends define an interior space for containing a drug reservoir. An impermeable member is positioned at the first end, said impermeable member preventing passage of the nucleic acid-based therapeutic agent out of the reservoir through the first end, and a permeable member is positioned at the second end, which allows diffusion of the nucleic acid-based therapeutic agent out of the reservoir through the second end.

The drug reservoir of such embodiments occupies a space defined by the tubular wall of the device and its termini. The reservoir may be filled with one or more fluid drug core compositions, including, but not limited to, solutions, suspensions, slurries, pastes, or other non-solid or semi-solid drug formulations containing a nucleic acid-based therapeutic agent. The reservoir may also be filled with a non-fluid (e.g., a gum, gel, or solid) drug core comprising at least one nucleic acid-based therapeutic agent.

In any event, it will be appreciated that as the nucleic acid-based therapeutic agent is released from the device over time, a non-fluid drug core that physically erodes as the agent dissolves away will not continue to fully occupy the reservoir volume. Applicants have found that a tube that has dimensional stability and is capable of supporting its own weight can accept a drug core therein without changing shape, and retain its structural integrity as the agent is released. Because the reservoir is defined by a relatively rigid tubular shell, the reservoir will maintain its shape and size, and so the regions of the device through which agent diffusion takes place will not change in area. As described in the equations below, constant diffusion area favors a constant rate of agent release.

The use of a sufficiently rigid tube of material to hold the drug reservoir during manufacture also makes for significantly easier handling of the tube and reservoir, because the tube fully supports both its own weight and the weight of the reservoir even when the reservoir is not solid. The pre-formed tube used in the present invention is not a simple coating, because a coating is typically not pre-formed and cannot support its own weight. Also, the rigid structure of such embodiments allows the use of slurries drawn into the tube, which facilitates the fabrication of longer cylindrical devices. Furthermore, because of the relative ease of manufacturing devices in accordance with such embodiments, more than one reservoir, optionally containing more than one agent, can be incorporated into a single device.

During use the invention, although the size and/or shape of the drug core may change as agent dissolves and diffuses out of the device, the tube that defines the volume of the drug reservoir is sufficiently strong or rigid to maintain a substantially constant diffusion area, so that the diffusion rate from the device does not change substantially despite dimensional changes in the drug core. By way of example and not of limitation, an exemplary method of ascertaining if the tube is sufficiently rigid is to form a device in accordance with the present invention, and to measure the diffusion rate of the agent from the device over time. If the diffusion rate changes more than 50% from the diffusion rate expected based on the chemical potential gradient across the device at any particular time, the tube has changed shape and is not sufficiently rigid. Another exemplary test is to visually inspect the device as the agent diffuses over time, looking for signs that the tube has collapsed in part or in full.

The use of permeable and impermeable tubes in accordance with the present invention provides resistance to reverse flow, i.e., flow back into the device. The tube or tubes assist in preventing large proteins from binding, solubilizing, or degrading the nucleic acid-based therapeutic agent before it leaves the drug reservoir. Also, the tube or tubes assist in preventing oxidation and protein lysis, as well as preventing other biological agents from entering the reservoir and degrading the contents.

It will be understood that “reservoir” generally refers to the inner volume of the device in the sense that it acts as a container, and “core” generally refers to the contents of the container. However, the terms “core” and “reservoir” are occasionally used interchangeably in describing the devices of the invention, because as initially manufactured the drug core and the drug reservoir that contains it are essentially co-extensive. As the device delivers the nucleic acid-based therapeutic agent during use, however, a solid drug core may gradually erode, and no longer be co-extensive with the drug reservoir that contains it.

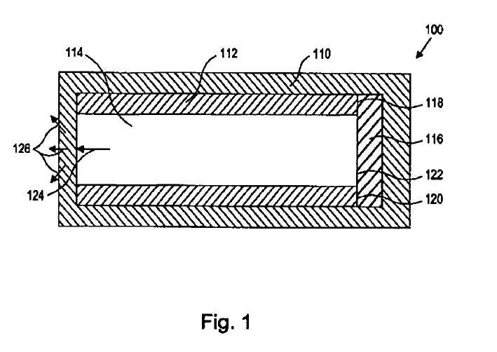

Turning now to the drawing figures, FIG. 1 illustrates a longitudinal cross-sectional view of a drug delivery device 100 in accordance with the present invention. Device 100 includes an outer layer 110, an inner tube 112, a reservoir or drug core 114, and an inner cap 116. Outer layer 110 is preferably a permeable layer, that is, the outer layer is permeable to the nucleic acid-based therapeutic agent contained within reservoir 114. Cap 116 is positioned at one end of tube 112. Cap 116 is preferably formed of an impermeable material, that is, the cap is not permeable to the nucleic acid-based therapeutic agent contained within reservoir 114. Cap 116 is joined at end 118, 120 of inner tube 112, so that the cap and the inner tube together close off a space in the tube in which reservoir 114 is positioned. Inner tube 112 and cap 116 can be formed separately and assembled together, or the inner tube and the cap can be formed as a single, integral, monolithic element.

Outer layer 110 at least partially, and preferably completely, surrounds both tube 112 and cap 116, as illustrated in FIG. 1. While it is sufficient for outer layer 110 to only partially cover tube 112 and cap 116, and in particular the opposite ends of device 100, the outer layer is preferably formed to completely envelop both the tube and cap to provide structural integrity to the device, and to facilitate further manufacturing and handling because the device is less prone to break and fall apart. While FIG. 1 illustrates cap 116 having an outer diameter the same as the outer diameter of inner tube 112, the cap can be sized somewhat smaller or larger than the outer diameter of the inner tube while remaining within the spirit and scope of such embodiments of the present invention.

Reservoir 114 is positioned inside inner tube 112, as described above. A first end 122 abuts against cap 116, and is effectively sealed by the cap against the diffusion of agent through the first end. On the end of reservoir 114 opposite cap 116, the reservoir is preferably in direct contact with outer layer 110. As will be readily appreciated by one of ordinary skill in the art, as nucleic acid-based therapeutic agent is released from a non-fluid core contained within reservoir 114, the core may shrink or otherwise change shape, and therefore may not fully or directly contact outer layer 110 at the end of the reservoir opposite cap 116. As outer layer 110 is permeable to the nucleic acid-based therapeutic agent in reservoir 114, the agent is free to diffuse out of the reservoir along a first flow path 124 into portions of outer layer 110 immediately adjacent to the open end of the reservoir. From outer layer 110, the agent is free to diffuse along flow paths 126 out of the outer layer and into the tissue or other anatomical structure in which device 100 is inserted. Optionally, holes can be formed through inner layer 112 to add additional flow paths 126 between reservoir 114 and permeable outer layer 110.

FIG. 1 illustrates only the positions of the several components of device 100 relative to one another, and for ease of illustration shows outer layer 110 and inner tube 112 as having approximately the same wall thickness. The thickness of the layer and wall are exaggerated for ease of illustration, and are not drawn to scale. While the walls of outer layer 110 and inner tube 112 may be of approximately the same thickness, the inner tube's wall thickness can be significantly thinner or thicker than that of the outer layer within the spirit and scope of the present invention. Additionally, device 100 is preferably cylindrical in shape, for which a transverse cross-section (not illustrated) will show a circular cross-section of the device. While it is preferred to manufacture device 100 as a cylinder with circular cross-sections, it is also within the scope of the invention to provide cap 116, nucleic acid-based therapeutic agent reservoir 114, inner tube 112, and/or outer layer 110 with other cross-sections, such as ovals, ellipses, rectangles, including squares, triangles, as well as any other regular polygon or irregular shapes. Furthermore, device 100 can optionally further include a second cap (not illustrated) on the end opposite cap 116; such a second cap could be used to facilitate handling of the device during fabrication, and would include at least one through hole for allowing nucleic acid-based therapeutic agent from reservoir 114 to flow from the device. Alternatively, the second cap may be formed of a permeable material.

Where the device is adapted for insertion into the lacrimal canaliculus, inner tube 112, 212, or 312 will be sized to fit within the lacrimal canaliculus, and will preferably be formed with a collarette, sized to rest on the exterior of the lacrimal punctum, at the end opposite cap 116, 242, or 316. It will be appreciated that permeable outer layer 110, 210, or 310 need not cover the entire device in this embodiment, as agent release will preferably be limited to the region of the device intended to remain external to the canaliculus.

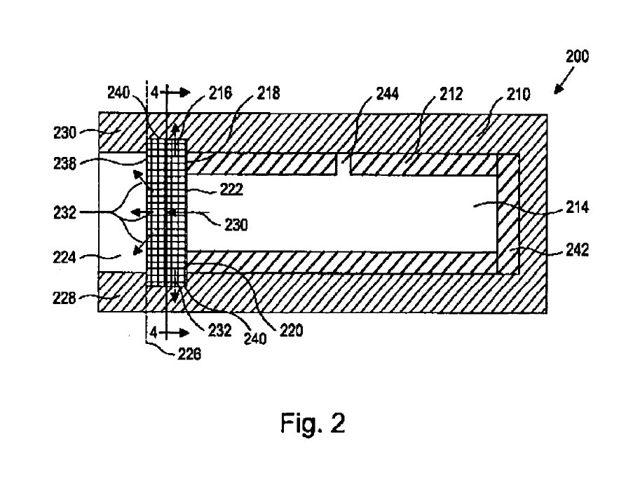

FIG. 2 illustrates a device 200 in accordance with a second example of such embodiments of the present invention. Device 200 includes an impermeable inner tube 212, a nucleic acid-based therapeutic agent drug core 214, and a permeable plug 216. Device 200 optionally and preferably includes an impermeable outer layer 210, which adds mechanical integrity and dimensional stability to the device, and aids in manufacturing and handling the device. As illustrated in FIG. 2, drug core 214 is positioned in the interior of inner tube 212, in a fashion similar to core 114 and inner tube 112 described above. Plug 216 is positioned at one end of inner tube 212, and is joined to the inner tube at end 218, 220 of the inner tube. While plug 216 may extend radially beyond inner tube 212, as illustrated in FIG. 2, the plug may alternatively have substantially the same radial extent as, or a slightly smaller radial extent than, the inner tube, while remaining within the scope of the invention. As plug 216 is permeable to the nucleic acid-based therapeutic agent contained in the reservoir, the nucleic acid-based therapeutic agent is free to diffuse through the plug from the reservoir. Plug 216 therefore must have a radial extent that is at least as large as the radial extent of reservoir 214, so that the primary diffusion pathway 230 out of the reservoir is through the plug. On the end of inner tube 212 opposite plug 216, the inner tube is closed off or sealed only by outer layer 210, as described below. Optionally, an impermeable cap 242, which can take the form of a disc, is positioned at the end of reservoir opposite plug 216. When provided, cap 242 and inner tube 212 can be formed separately and assembled together, or the inner tube and the cap can be formed as a single, integral, monolithic element.

Outer tube or layer 210, when provided, at least partially, and preferably completely, surrounds or envelopes inner tube 212, nucleic acid-based therapeutic agent reservoir 214, plug 216, and optional cap 242, except for an area immediately adjacent to the plug which defines a port 224. Port 224 is, in preferred embodiments, a hole or blind bore which leads to plug 216 from the exterior of the device. As outer layer 210 is formed of a material that is impermeable to the nucleic acid-based therapeutic agent in reservoir 214, the ends of inner tube 212 and reservoir 214 opposite plug 216 are effectively sealed off, and do not include a diffusion pathway for the nucleic acid-based therapeutic agent to flow from the reservoir. According to a preferred embodiment, port 224 is formed immediately adjacent to plug 216, on an end 238 of the plug opposite end 222 of reservoir 214. Plug 216 and port 224 therefore include diffusion pathways 230, 232, through the plug and out of device 200, respectively.

While port 224 in the embodiment illustrated in FIG. 2 has a radial extent that is approximately the same as inner tube 212, the port can be sized to be larger or smaller, as will be readily apparent to one of ordinary skill in the art. For example, instead of forming port 224 radially between portions 228, 230 of outer layer 210, these portions 228, 230 can be removed up to line 226, to increase the area of port 224. Port 224 can be further enlarged, as by forming outer layer 210 to extend to cover, and therefore seal, only a portion or none of the radial exterior surface 240 of plug 216, thereby increasing the total surface area of port 224 to include a portion or all of the outer surface area of the plug.

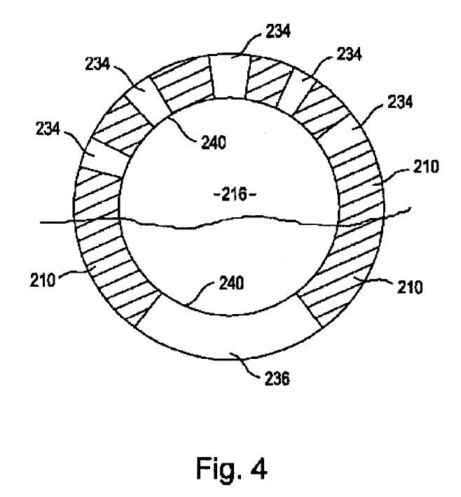

In accordance with yet another embodiment of the invention, port 224 of device 200 can be formed immediately adjacent to radial external surface 240 of plug 216, in addition to or instead of being formed immediately adjacent to end 238 of the plug. As illustrated in FIG. 4, port 224 can include portions 234, 236, which extend radially away from plug 216. These portions can include large, continuous, circumferential and/or longitudinal portions 236 of plug 216 which are not enveloped by outer layer 210, illustrated in the bottom half of FIG. 4, and/or can include numerous smaller, circumferentially spaced apart portions 234, which are illustrated in the top half of FIG. 4. Advantageously, providing port 224 immediately adjacent to radial external surface 240 of plug 216, as numerous, smaller openings 234 to the plug, allows numerous alternative pathways for the nucleic acid-based therapeutic agent to diffuse out of device 200 in the event of a blockage of portions of the port. Larger openings 236, however, benefit from a relative ease in manufacturing, because only a single area of plug 216 need be exposed to form port 224.

According to yet another embodiment of the invention, plug 216 is formed of an impermeable material and outer layer 210 is formed of a permeable material. A hole or holes are formed, e.g., by drilling, through one or more of inner layer 212, cap 242, and plug 216, which permit nucleic acid-based therapeutic agent to be released from reservoir 214 through outer layer 210. According to another embodiment, plug 216 is eliminated as a separate member, and permeable outer layer 210 completely envelopes inner tube 212 and cap 242 (if provided). Thus, the diffusion pathways 230, 232 are through outer layer 210, and no separate port, such as port 224, is necessary. By completely enveloping the other structures with outer layer or tube 210, the system 200 is provided with further dimensional stability. Further optionally, plug 216 can be retained, and outer layer 210 can envelop the plug as well.

According to yet another such embodiment of the present invention, inner tube 212 is formed of a permeable material, outer layer 210 is formed of an impermeable material, and cap 242 is formed of either a permeable or an impermeable material. Optionally, cap 242 can be eliminated. As described above, as outer layer 210 is impermeable to the nucleic acid-based therapeutic agent in reservoir 214, plug 216, port 224, and optional ports 234, 236, are the only pathways for passage of the nucleic acid-based therapeutic agent out of device 200.

The shape of device 200 can be, in a manner similar to that described above with respect to device 100, any of a large number of shapes and geometries. Furthermore, both device 100 and device 200 can include more than one reservoir 114, 214, included in more than one inner tube 112, 212, respectively, which multiple reservoirs can include different nucleic acid-based therapeutic agents, or ocular medicaments such as a miotic agent, beta-blocker or alpha agonist in addition to a nucleic acid-based therapeutic agent, for diffusion out of the device. In device 200, multiple reservoirs 214 can be positioned to abut against only a single plug 216, or each reservoir 214 can have a dedicated plug for that reservoir. Such multiple reservoirs can be enveloped in a single outer layer 110, 210, as will be readily appreciated by one of ordinary skill in the art.

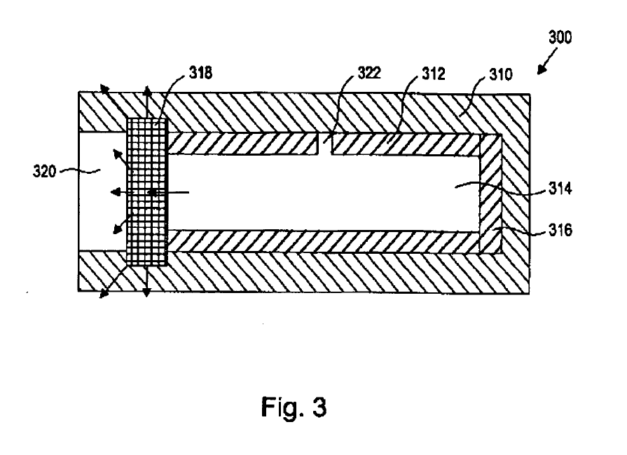

Turning now to FIG. 3, FIG. 3 illustrates a device 300 in accordance with a third exemplary embodiment of the invention. Device 300 includes a permeable outer layer 310, an impermeable inner tube 312, a reservoir 314, an impermeable cap 316, and a permeable plug 318. A port 320 communicates plug 318 with the exterior of the device, as described above with respect to port 224 and plug 216. Inner tube 312 and cap 316 can be formed separately and assembled together, or the inner tube and the cap can be formed as a single, integral, monolithic element. The provision of permeable outer layer 310 allows the nucleic acid-based therapeutic agent in reservoir or drug core 314 to flow through the outer layer in addition to port 320, and thus assists in raising the overall delivery rate. Of course, as will be readily appreciated by one of ordinary skill in the art, the permeability of plug 318 is the primary regulator of the agent delivery rate, and is accordingly selected. Additionally, the material out of which outer layer 310 is formed can be specifically chosen for its ability to adhere to the underlying structures, cap 316, tube 312, and plug 318, and to hold the entire structure together. Optionally, a hole or holes 322 can be provided through inner tube 312 to increase the flow rate of nucleic acid-based therapeutic agent from reservoir 314.

In order to maximize the useful life of the device, preferred formulations will be those that contain as large a mass of active agent as possible while retaining an effective rate of dissolution. By way of example, a dense, compressed solid that contains at least 90% of a non-salt form of a nucleic acid-based therapeutic agent would be a preferred drug core formulation.

A large number of materials can be used to construct the devices of the present invention. The only requirements are that they are inert, non-immunogenic, and of the desired permeability, as described herein.

In another embodiment, only a single outer layer need be used. FIG. 6 illustrates such an embodiment, wherein the sustained release device (product 612) includes an outer layer or skin 614 and an inner core 616.

Materials that may be suitable for fabricating devices 100, 200, 300, and 712 include naturally occurring or synthetic materials that are biologically compatible with body fluids and/or eye tissues, and essentially insoluble in body fluids with which the material will come in contact. The use of rapidly dissolving materials or materials highly soluble in eye fluids are to be avoided since dissolution of the outer layers 110, 210, 310 would affect the constancy of the agent release, as well as the capability of the system to remain in place for a prolonged period of time.

Naturally occurring or synthetic materials that are biologically compatible with body fluids and eye tissues and essentially insoluble in body fluids with which the material will come in contact include, but are not limited to: ethyl vinyl acetate, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasticized ethylene vinylacetate copolymer, polyvinyl alcohol, ethylene vinylchloride copolymer, polyvinyl esters, polyvinylbutyrate, polyvinylformal, polyamides, poly(methyl methacrylate), poly(butyl methacrylate), plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinyl chloride-diethyl fumarate copolymer, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer, vinylidene chloride-acrylonitrile copolymer, gold, platinum, and (surgical) stainless steel.

Specifically, outer layer 210 of device 200 may be made of any of the above-listed polymers or any other polymer that is biologically compatible with body fluids and eye tissues, essentially insoluble in body fluids with which the material will come in contact, and permeable to the passage of the nucleic acid-based therapeutic agent.

When inner tube 112, 212, 312 is selected to be impermeable, as described above, to the passage of the nucleic acid-based therapeutic agent from the inner core or reservoir out to adjacent portions of the device, the purpose is to block the passage of the nucleic acid-based therapeutic agent through those portions of the device, and thus limit the release of the nucleic acid-based therapeutic agent from the device to selected regions of the outer layer and plugs 216 and 318.

The composition of outer layer 110, e.g., the polymer, is preferably selected so as to allow the above-described controlled release. The preferred composition of outer layer 110 and plug 216 will vary depending on such factors as the identity of the nucleic acid-based therapeutic agent, the desired rate of release, and the mode of implantation or insertion. The identity of the active agent is important since it determines the desired therapeutic concentration, and because the physico-chemical properties of the molecule are among the factors that affect the rate of release of the agent into and through the outer layer 110 and plug 216.

Caps 116, 242, 316 are impermeable to the passage of the nucleic acid-based therapeutic agent and may cover a portion of the inner tube not covered by the outer layer. The physical properties of the material, preferably a polymer, used for the caps can be selected based on their ability to withstand subsequent processing steps (such as heat curing) without suffering deformation of the device. The material, e.g., polymer, for impermeable outer layer 210 can be selected based on the ease of coating inner tube 212. Cap 116 and inner tubes 112, 212, 312 can independently be formed of any of a number of materials, including PTFE, polycarbonate, polymethyl methacrylate, polyethylene alcohol, high grades of ethylene vinyl acetate (9% vinyl, content), and polyvinyl alcohol (PVA). Plugs 216, 318 can be formed of any of a number of materials, including cross-linked PVA, as described below.

Outer layers 110, 210, 310, and plugs 216, 318 of the device must be biologically compatible with body fluids and tissues, essentially insoluble in body fluids with which the material will come in contact, and outer layer 110 and plugs 216, 318 must be permeable to the passage of the nucleic acid-based therapeutic agent.

The nucleic acid-based therapeutic agent diffuses in the direction of lower chemical potential, i.e., toward the exterior surface of the device. At the exterior surface of the device, equilibrium is again established. When the conditions on both sides of outer layer 110 or plugs 216, 318 are maintained constant, a steady state flux of the nucleic acid-based therapeutic agent will be established in accordance with Fick's Law of Diffusion. The rate of passage of the agent through the material by diffusion is generally dependent on the solubility of the agent therein, as well as on the thickness of the wall. This means that selection of appropriate materials for fabricating outer layer 110 and plug 216 will be dependent on the particular nucleic acid-based therapeutic agent to be used.

The rate of diffusion of the nucleic acid-based therapeutic agent through a polymeric layer of the invention may be determined via diffusion cell studies carried out under sink conditions. In diffusion cell studies carried out under sink conditions, the concentration of agent in the receptor compartment is essentially zero when compared to the high concentration in the donor compartment. Under these conditions, the rate of agent release is given by:
Q/t=(D·K·A·DC)/h
where Q is the amount of agent released, t is time, D is the diffusion coefficient, K is the partition coefficient, A is the surface area, DC is the difference in concentration of the agent across the membrane, and h is the thickness of the membrane.

In the case where the agent diffuses through the layer via water filled pores, there is no partitioning phenomenon. Thus, K can be eliminated from the equation. Under sink conditions, if release from the donor side is very slow, the value DC is essentially constant and equal to the concentration of the donor compartment. Release rate therefore becomes dependent on the surface area (A), thickness (h), and diffusivity (D) of the membrane. The surface area is a function of the size of the particular device, which in turn is dependent on the desired size of the drug core or reservoir.

Thus, permeability values may be obtained from the slopes of a Q versus time plot. The permeability P, can be related to the diffusion coefficient D, by:
P=(K·D)/h

Once the permeability is established for the material permeable to the passage of the agent, the surface area of the agent that must be coated with the material impermeable to the passage of the agent may be determined. This may be done by progressively reducing the available surface area until the desired release rate is obtained.

Exemplary microporous materials suitable for use as outer layer 110 and plugs 216, 318, for instance, are described in U.S. Pat. No. 4,014,335, which is incorporated herein by reference in its entirety. These materials include but are not limited to cross-linked polyvinyl alcohol, polyolefins or polyvinyl chlorides or cross-linked gelatins; nylon, regenerated, insoluble, non-erodable cellulose, acylated cellulose, esterified celluloses, cellulose acetate propionate, cellulose acetate butyrate, cellulose acetate phthalate, cellulose acetate diethyl-aminoacetate; polyurethanes, polycarbonates, and microporous polymers formed by co-precipitation of a polycation and a polyanion modified insoluble collagen. Cross-linked polyvinyl alcohol is preferred for both outer layer 110 and plugs 216, 318. Preferred impermeable portions of the devices, e.g., cap 116 and inner tubes 112, 212, are formed of PTFE, ethyl vinyl alcohol, polyimide, or silicone.

The drug delivery system of the present invention may be inserted into or adjacent to the eye via any of the methods known in the art for ocular implants and devices. One or more of the devices may be administered at one time, or more than one agent may be included in the inner core or reservoir, or more than one reservoir may be provided in a single device.

Devices intended for insertion into the eye, for example into the vitreous chamber, may remain in the vitreous permanently after treatment is complete. Such devices may provide sustained release of the nucleic acid-based therapeutic agent for a period of from several days to over five years. In certain embodiments, sustained release of the at least one agent may occur for a period of one or more months, or even greater than one or more years.

When such devices are prepared for insertion within the vitreous of the eye, it is preferred that the device does not exceed about 7 millimeters in any direction. Thus, the cylindrical devices illustrated in FIGS. 1 and 2 would preferably not exceed 7 millimeters in height or 3 millimeters in diameter, and are more preferably less than 1 mm in diameter and most preferably less than 0.5 mm in diameter. The preferred thickness of the walls of inner tubes 112, 212 ranges between about 0.01 mm and about 1.0 mm. The preferred thickness of the wall of outer layer 110 ranges between about 0.01 mm and about 1.0 mm. The preferred thickness of the wall of outer layer 210 ranges between about 0.01 mm and 1.0 mm. The inner agent-containing core of the various embodiments of the present invention preferably contains a high proportion of nucleic acid-based therapeutic agent, so as to maximize the amount of agent contained in the device and maximize the duration of agent release. Accordingly, in some embodiments, the drug core may consist entirely of one or more nucleic acid-based therapeutic agents in crystalline or amorphous form.

As noted above, the nucleic acid-based therapeutic agent may be present in neutral form, or it may be in the form of a pharmaceutically acceptable salt, a codrug, or a prodrug. Where the nucleic acid-based therapeutic agent comprises less than 100% of the core, suitable additives that may be present include, but are not limited to, polymeric matrices (e.g., to control dissolution rate or to maintain the shape of the core during use), binders (e.g., to maintain the integrity of the core during manufacture of the device), and additional pharmacological agents.

In some embodiments, the inner core is solid and is compressed to the highest density feasible, again to maximize the amount of contained agent. In alternative embodiments, the drug core may not be solid. Non-solid forms include, but are not limited to, gums, pastes, slurries, gels, solutions, and suspensions. It will be appreciated that the drug core may be introduced to the reservoir in one physical state and thereafter assume another state (e.g., a solid drug core may be introduced in the molten state, and a fluid or gelatinous drug core may be introduced in a frozen state).

While the above described embodiments of the invention are described in terms of preferred ranges of the amount of effective agent, and preferred thicknesses of the preferred layers, these preferences are by no means meant to limit the invention. As would be readily understood by one skilled in the art, the preferred amounts, materials and dimensions depend on the method of administration, the effective agent used, the polymers used, the desired release rate and the like. Likewise, the desired release rates and duration of release depend on a variety of factors in addition to the above, such as the disease state being treated, the age and condition of the patient, the route of administration, and other factors which would be readily apparent to those skilled in the art.

From the foregoing description, one of ordinary skill in the art can easily ascertain the essential characteristics of the instant invention, and without departing from the spirit and scope thereof, can make various changes and/or modifications of the invention to adapt it to various usages and conditions. As such, these changes and/or modifications are properly, equitably and intended to be, within the full range of equivalence of the following claims.

All publications and patents mentioned herein are hereby incorporated by reference in their entirety, as if each individual publication or patent was specifically and individually indicated to be incorporated by reference.

1. A controlled- and sustained-release drug delivery device comprising
(a) an inner drug core comprising an amount of a nucleic acid-based therapeutic agent, and
(b) a first polymer coating partially covering said core,
wherein said polymer coating is impermeable to said therapeutic agent.

2. The drug delivery device of claim 1, further comprising a second polymer coating covering at least the portion of said core not covered by said first polymer layer, wherein the second polymer coating is permeable to the therapeutic agent.

3. The drug delivery device of claim 2, wherein the second polymer layer is positioned between the core and the first polymer layer.

4. The drug delivery device of claim 2, wherein the first polymer layer is positioned between the core and the second polymer layer.

5. A controlled and sustained release drug delivery device comprising
(a) an inner drug core comprising an amount of a nucleic acid-based therapeutic agent,
(b) an inner tube impermeable to the passage of said agent, said inner tube having first and second ends and covering at least a portion of said inner drug core, said inner tube being dimensionally stable,
(c) an impermeable member positioned at said inner tube first end, said impermeable member preventing passage of said agent out of said drug core through said inner tube first end, and
(d) a permeable member positioned at said inner tube second end, said permeable member allowing diffusion of said agent from said drug core through said inner tube second end.

6. A controlled and sustained release drug delivery device comprising
(e) an inner drug core comprising an amount of a nucleic acid-based therapeutic agent,
(f) an inner tube impermeable to the passage of said agent, said inner tube having first and second ends and covering at least a portion of said inner drug core, said inner tube being dimensionally stable, and
(g) permeable members positioned at said inner tube first and second ends, said permeable members allowing diffusion of said agent from said drug core through said inner tube first and second ends.

7. A controlled- and sustained-release drug delivery device comprising
(a) a drug core comprising an amount of a nucleic acid-based therapeutic agent,
(b) a first polymer coating permeable to the passage of said agent, and
(c) a second polymer coating impermeable to the passage of said agent,
wherein the second polymer coating covers a portion of the surface area of the drug core and/or the first polymer coating.

8. A controlled- and sustained-release drug delivery device comprising
(a) a drug core comprising an amount of a nucleic acid-based therapeutic agent, and
(b) a first polymer coating and a second polymer coating permeable to the passage of said agent,
wherein the two polymer coatings are bioerodable and erode at different rates.

9. A controlled- and sustained-release drug delivery device comprising
(a) a drug core comprising an amount of a nucleic acid-based therapeutic agent,
(b) a first polymer coating permeable to the passage of said agent covering at least a portion of the drug core,
(c) a second polymer coating impermeable to the passage of said agent covering at least a portion of the drug core or the first polymer coating, and
(d) a third polymer coating permeable to the passage of said agent covering the drug core and the second polymer coating,
wherein a dose of said agent is released for at least 7 days.

10. A controlled- and sustained-release drug delivery device comprising
(a) a drug core comprising an amount of a nucleic acid-based therapeutic agent,
(b) a first polymer coating permeable to the passage of said agent covering at least a portion of the drug core,
(c) a second polymer coating impermeable to the passage of said agent covering at least a portion of the drug core or the first polymer coating, and
(d) a third polymer coating permeable to the passage of said agent covering the drug core and the second polymer coating,
wherein release of said agent maintains a desired concentration of said agent for at least 7 days.

11. A controlled- and sustained-release drug delivery device comprising
(a) a drug core comprising an amount of a nucleic acid-based therapeutic agent, and
(b) a non-erodable polymer coating, the polymer coating being permeable to the passage of said agent covering the drug core and essentially non-release rate limiting,
wherein a dose of said agent is released for at least 7 days.

12. A controlled- and sustained-release drug delivery device comprising
(a) a drug core comprising an amount of a nucleic acid-based therapeutic agent, and
(b) a non-erodable polymer coating, the polymer coating being permeable to the passage of said agent covering the drug core and being essentially non-release rate limiting,
wherein release of said agent maintains a desired concentration of said agent for at least 7 days.

13. A controlled- and sustained-release drug delivery device comprising
(a) a drug core comprising an amount of a nucleic acid-based therapeutic agent,
(b) a first polymer coating permeable to the passage of said agent covering at least a portion of the drug core,
(c) a second polymer coating impermeable to the passage of said agent covering at least 50% of the drug core and/or the first polymer coating, said second polymer coating comprising an impermeable film and at least one impermeable disc, and
(d) a third polymer coating permeable to the passage of said agent covering the drug core, the uncoated portion of the first polymer coating, and the second polymer coating,
wherein a dose of said agent is released for at least 7 days.

14. A controlled- and sustained-release drug delivery device comprising
(a) a drug core comprising an amount of a nucleic acid-based therapeutic agent,
(b) a first polymer coating permeable to the passage of said agent covering at least a portion of the drug core,
(c) a second polymer coating impermeable to the passage of said agent covering at least 50% of the drug core and/or the first polymer coating, said second polymer coating comprising an impermeable film and at least one impermeable disc, and
(d) a third polymer coating permeable to the passage of said agent covering the drug core, the uncoated portion of the first polymer coating, and the second polymer coating,
wherein release of said agent maintains a desired concentration of said agent for at least 7 days.

15. The device according to claim 1, wherein the first polymer coating comprises polyimide, silicone, poly(lactic acid), poly(lactic-co-glycolic acid), or poly(caprolactone).

16. The device according to claim 1, wherein the second polymer coating comprises cross-linked polyvinyl alcohol, poly(lactic acid), poly(lactic-co-glycolic acid), or poly(caprolactone).

17. The device according to claim 1, wherein the second polymer coating further comprises polyethylene glycol.

18. The device according to claim 16, wherein the second polymer coating further comprises polyethylene glycol.

19. A device according to claim 1, wherein the nucleic acid-based therapeutic agent is an aptamer.

20. A device according to claim 1, wherein the nucleic acid-based therapeutic agent is a ribozyme.

21. A device according to claim 1, wherein the nucleic acid-based therapeutic agent is an antisense agent.

22. A device according to claim 1, wherein the nucleic acid-based therapeutic agent is a small inhibitory RNA.

23. A device according to claim 19, wherein the nucleic acid-based therapeutic agent is pegaptanib.

24. A device according to claim 20, wherein the nucleic acid-based therapeutic agent is Angiozyme™.

25. A device according to claim 21, wherein the nucleic acid-based therapeutic agent is selected from fomivirsen, alicaforsen, oblimersen, Affinitac™, and Oncomyc-NG™.
